(19) United States
(12) Patent Application Publication
(10) Pub. No.: US 2019/0071662 A1
(43) Pub. Date: Mar. 7, 2019

(54) Methods for the Isolation of Biomolecules and Uses Thereof

(71) Applicant: New York Genome Center, Inc., New York, NY (US)

(72) Inventor: Brian Houck-Loomis, New York, NY (US)

(21) Appl. No.: 16/093,627

(22) PCT Filed: Apr. 13, 2017
(86) PCT No.: PCT/US2017/027459

§ 371 Date: Oct. 13, 2018

Related U.S. Application Data
(63) Continuation-in-part of application No. 15/097,781, filed on Apr. 13, 2016.

(51) Int. Cl. C12N 15/10; G01N 1/34; C12Q 1/6806; G01N 1/40; C12Q 1/6874

(52) U.S. Cl. CPC C12N 15/101; G01N 1/34; C12Q 2527/119; G01N 1/40; C12Q 1/6874; C12Q 1/6806

(57) ABSTRACT

Methods for preparing and utilizing tunable electrostatic capture (“TEC”) ligands that have an ionizable function are provided. The electrostatic nature of the ionizable functionality of the TEC ligands can be “tuned” or adjusted to either reversibly bind or release a desired target anion, such as a biomolecule, by varying the pH and/or the ionic strength of the binding conditions and release conditions. The TEC ligands can be bound to a solid support to form TEC binding surfaces. TEC surfaces, ligands, solid supports and the accompanying methods and buffer systems can be used to isolate polyanions, such as nucleic acids, from materials, for example in a size-selective manner.

### CROSS-REFERENCE TO RELATED APPLICATIONS

This application is a continuation-in-part application of, and claims priority of, U.S. patent application Ser. No. 15/097,781, filed Apr. 13, 2016, which application is incorporated herein by reference.

### BACKGROUND OF THE INVENTION

A crucial advance in modern biology and its clinical applications is the ability to purify specific molecules of interest from a range of biological materials. There are currently many methods that can be used for the isolation of biomolecules, which vary in the yield, scalability, reproducibility, quality, integrity, and purity of the biomolecules isolated.

Nucleic acid sequencing is one of the most widely used tools in molecular biology. Development of rapid and sensitive sequencing methods utilizing automated DNA sequencers has revolutionized modem molecular biology. In particular, analysis of entire genomes of plants, viruses, bacteria, fungi, and animals is now possible with next-generation sequencing technology.

The process of preparing DNA for sequencing requires the ability to efficiently discriminate between the sizes of nucleic acid molecules within a biological sample, especially at the upper or lower size ranges. The current methods that allow for nucleic acid size selection tend to be costly and time-consuming. As a result, whole-genome sequencing continues to be expensive and labor intensive because the related work flows tend to be complex, time-consuming, and costly to perform. For example, systems for the rapid and efficient isolation and size separation of nucleic acids prior to cloning and/or sequencing were previously not available. Additionally, current methods were limited in their ability to efficiently discriminate between the sizes of nucleic acid molecules within a biological sample, especially at the upper or lower size ranges.

For example, a common workflow for generating next-generation sequencing molecules, or “libraries”, utilizes solid phase reversible immobilization (“SPRI”) beads having carboxyl groups on the bead surface that reversibly bind nucleic acids in the presence of polyethylene glycol (PEG) and salt.

SPRI beads are commonly used for low concentration DNA cleanup. Additionally, SPRI beads can be used, to an extent, in nucleic acid size-selection protocols by varying the PEG concentration. The concentration of PEG and salt, changed by altering the ratio of SPRI bead solution:DNA, influences the sizes of nucleic acid fragments that bind to or elute from the beads. As this ratio changes, the lengths of nucleic acid fragments bound and/or left in solution also changes. A lower ratio of SPRI:DNA correlates with the capture and elution of larger final fragments.

Due to the viscous nature of PEG, precipitation of DNA onto the SPRI beads and magnetic collection of the beads is time-consuming. Additionally, the ability to discriminate between sizes of nucleic acids using the SPRI bead/PEG methods is limited. It is not possible to discriminate between nucleic acid fragments having sizes less than 35 base pairs (bp). Accordingly, the SPRI bead/PEG methods do not fulfill the need for systems with rapid and efficient nucleic acid size selection capabilities.

Other methods for isolating nucleic acids from a biological sample are similarly slow and cumbersome, and do not fulfill the need for systems with rapid and efficient size-selection capabilities. For example, CHARGESWITCH® technology, which is described in U.S. Pat. No. 6,914,137, relates to a method for extracting nucleic acids from a biological sample by contacting the sample with a material at a pH such that the material is positively charged and will bind negatively charged nucleic acids. The nucleic acids are released at a pH when the materials possess a neutral or negative charge. However, it is not possible to use the CHARGESWITCH® technology to perform meaningful size selection of nucleic acids. Accordingly, the CHARGESWITCH® technology also fails to provide a system with rapid and efficient nucleic acid size-selection capabilities.

### SUMMARY OF THE INVENTION

This disclosure is directed, in part, toward preparing and utilizing tunable electrostatic capture (“TEC”) ligands and surfaces to isolate polyanions, such as nucleic acids, from materials or samples, including biological materials or samples.

In one aspect, a method for separating polyanions according to size from a sample comprises contacting a sample which comprises polyanions of different sizes with a solid support to which is covalently bound a plurality of tunable electrostatic capture (TEC) ligands. The TEC ligands comprise an ionizable ligand which is positively charged at a pH less than a pKa of the ionizable ligand and neutral at a pH greater than the pKa of the ionizable ligand. The support has been contacted with a TEC capture buffer having a pH less than the pKa of the ionizable ligand, which causes the TEC ligands to have a positive charge. The sample is contacted under conditions which allow the TEC ligands to reversibly bind polyanions of different sizes in the sample to the TEC ligands having a positive charge. Still another step involves releasing the polyanions from the TEC ligands according to size by contacting the solid support with at least a first TEC release buffer having an ion concentration suitable for releasing polyanions having a first size from the TEC ligands, wherein polyanions having a second size greater than the first size remain bound to the TEC ligands.

In another aspect, a method comprises the steps of: (a) preparing a TEC surface, (b) mixing a polyanion-containing sample with the TEC surface from step (a) in a defined buffer that promotes binding of the polyanion(s) of interest, and (c) adjusting the pH of the bound polyanion-TEC surface mixture by addition of a second defined buffer to promote elution of the polyanion from the TEC surface.

In another aspect, a rapid and efficient method for polyanion size selection comprises the steps of: (a) preparing a TEC surface, (b) mixing a polyanion-containing sample with the TEC surface from step (a) in a defined buffer that promotes binding of the polyanion(s) of interest, and (c) adjusting the ionic strength of the bound polyanion-TEC surface mixture by addition of a second defined buffer to promote elution of polyanions of a given size from the TEC surface, or (d) washing of the bound polyanion-TEC surface mixture with a sequence of buffers of increasing ionic strength to promote elution of polyanions from the TEC surface in a size selective manner.

In another aspect, a method is provided for normalizing a concentration of polyanions, for example, nucleic acids, from a plurality of samples, for example biologic samples. The method comprises providing a plurality of samples comprising polyanions, wherein at least one of the plurality of samples has a different concentration of polyanions than the other samples. Substantially the same amount of a solid support covalently bound to TEC ligands is mixed with each of the plurality of samples in liquid medium (also referred to herein as a TEC capture buffer) under substantially the same conditions to form a mixture, in which the mixture has a pH less than the pKa of the TEC ionizable ligand. The solid support covalently bound to TEC ligands is then recovered from each sample, and kept separate. A substantially similar amount of polyanions is then released from the TEC-ligands bound to the solid support recovered from each sample, by contacting the solid support with one or more buffers having a pH greater than the pKa of the ionizable ligand. The released polyanions can be reconstituted or diluted in a substantially similar amount of vehicle (such as a storage or sequencing buffer), thus resulting in a normalized concentration of polyanions recovered from each sample.

In still other aspects, kits are provided containing the components necessary to practice the described methods.

Still other objects and advantages of the invention will become apparent to those of skill in the art from the disclosure herein, which is simply illustrative and not restrictive. Thus, other embodiments will be recognized by the skilled artisan without departing from the spirit and scope of the invention.

### BRIEF DESCRIPTION OF THE FIGURES

FIG. 1 shows exemplary TEC ligands that feature a primary amine, which acts as a chemical ligation handle, and an aromatic amine moiety.

FIG. 2A is a diagram showing magnetic carboxylate beads passivated by treatment with acetic anhydride (Ac₂O) and diisopropyl ethyl amine (DIEA) in N,N-dimethyl formamide (DMF).

FIG. 2B is a diagram showing passivated surfaces conjugated with organic-soluble ligand containing a primary amine for conjugation and an aromatic amine moiety.

FIG. 2C is a diagram showing altering of the protonation state of the attached ligand, which depends on the pH of surrounding medium, and can be accomplished by washing the conjugated beads into aqueous buffers.

FIG. 2D shows the Henderson-Hasselbalch equation, which describes the relationship between solution pH and charge state of the resin. When the solution pH is equal to the pKa of the bound ligand, 50% of the heterocyclic amine is charged. At 1 pH unit below the pKa, 90% of the ligand is charged. At 1 pH unit above the pKa, 10% of the ligand is charged.

FIGS. 3A and 3B are diagrams showing a TEC ligand reversibly binding and releasing a DNA polyanion. FIG. 3A is a diagram of a TEC ligand bound to a solid support binding DNA polyanion by electrostatic capture. FIG. 3B is a diagram showing elution of bound DNA polyanions by means of electrostatic (charge) screening as an alternative to pH-induced elution.

FIG. 4 is a graph showing the average Ct value (n=3) of DNA recovered from TEC beads after being incubated in acidic binding buffer for 5, 15, 30, or 60 minutes. To determine Ct values, the DNA was mass normalized and subjected to qPCR.

FIG. 5A is a graph showing pH elution characterizations, i.e., pH profiles, for TEC ligand, passivated histamine and (2-aminoethyl)pyridine (AEP) resins.

FIG. 5B is a graph of percent recovery of DNA bound to various TEC resins washed with two different pH buffers (5.5 and 6.5), and eluted with buffer at pH 8.3.

FIG. 5C is a graph showing pH profiles for the TEC ligand, non-passivated histamine resins (beads).

FIG. 5D is a graph showing pH profiles of the TEC ligand, passivated histamine TEC resins (beads).

FIG. 5E is a graph showing pH profiles for the TEC ligand, non-passivated AEP resins (beads).

FIG. 5F is a graph showing pH profiles for passivated AEP resins (beads).

FIG. 6 shows a graph of the percent of DNA recovery with various TEC ligands to which purified DNA was bound at low pH, washed with intermediate pH (specific for each TEC ligand), and eluted at pH 8.3.

FIG. 7A is a graph showing comparison of TEC and SPRI technologies for post-PCR DNA recovery, i.e., showing concentration of DNA recovered post-PCR using TEC (Cap-His100) and SPRI methods.

FIG. 7B shows NanoDrop traces for the replicates in FIG. 7A.

FIG. 8 is a graph showing the RNA recovery, expressed as a percentage of input, for a standard RNA sample (Universal Human Reference, UHR) captured and purified with either SPRI beads or TEC resins (n=3).

FIG. 9 is a graph showing DNA eluted from Cap-His100 TEC resin (in ng/ml) at different buffer pH values.

FIG. 10A shows pH size selection elution from a TEC resin. Trace from on-chip electrophoresis (Agilent Bioanalyzer) showing sizes of DNA molecules eluted from TEC beads (Cap-His100) subjected to incremental pH washes from pH 6.55-6.95.

FIG. 10B shows pH size selection elution from a TEC resin. Trace from on-chip electrophoresis (Agilent Bioanalyzer) showing sizes of DNA molecules eluted from TEC beads (Cap-His100) using a narrow pH range (6.48-6.60).

FIG. 11A shows sodium chloride size selection of DNA molecules of discrete sizes from TEC resin. A recovery profile of a defined low-molecular weight DNA ladder from TEC resin (Cap-His100) is shown at increasing concentrations of ions (NaCl).

FIG. 11B shows sodium chloride size selection of DNA molecules of discrete sizes from TEC resin. A binding profile of a defined low-molecular weight DNA ladder to TEC resin (Cap-His100) is shown at increasing concentrations of ions (NaCl).

FIG. 12A shows sodium chloride size selection of a DNA smear from TEC resin. Atrace from on-chip electrophoresis (Agilent Bioanalyzer) shows recovery of a DNA fragment pool (35-1500 bp) eluted from TEC resin (Cap-His100) with increasing concentrations of NaCl at a constant pH (6.0).

FIG. 12B shows the same data as in FIG. 12A as a gel representation.

FIG. 13 is a trace from on-chip electrophoresis (Agilent Bioanalyzer) showing imidazole size selective elution of DNA bound to Cap His100 TEC resinat pH 6.

FIG. 14A shows a Fragment Analyzer (Advanced Analytics) size analysis trace of nucleic acids recovered from FFPE materials using the TEC Express and Covaris truXTRAC DNA FFPE extraction methods.

FIG. 14B is a trace from on-chip electrophoresis (Agilent Bioanalyzer) showing the recovery of small-size RNA recovered from FFPE materials using the TEC Express extraction method.

FIG. 15A shows sodium chloride size selection of a DNA Smear on 2AEP TEC Resin. Trace from on-chip electrophoresis (Agilent Bioanalyzer) shows recovery of a DNA fragment pool (35-1500 bp) eluted from TEC resin (Cap-2AEP100) with increasing concentrations of NaCl at a constant pH (5.0).

FIG. 15B shows the same data as in FIG. 15A as a gel representation.

FIG. 16 is a graph showing a comparison of the capture capabilities of carboxy modified (CM), 2-Aminoethylpyridine-modified resin (Cap-2AEP100), and histimine-modified resin (Cap-His100) beads, expressed as a percentage of DNA recovered.

FIG. 17A shows the size selection of carboxy-modified beads without TEC ligands (CM beads). An elution profile of DNA is shown from CM beads which are not conjugated with any TEC ligands, eluted with increasing NaCl concentrations (0 to 500 mM).

FIG. 17B shows a trace from on-chip electrophoresis (Agilent Bioanalyzer) showing recovery of a DNA fragment pool eluted from CM beads of FIG. 17A with increasing NaCl concentrations (0 to 500 mM) at pH 4.5 and 5.0.

FIG. 18 is a graph showing the amount of DNA recovered (in ng/μl) from TEC and CHARGESWITCH® beads using buffers appropriate to each method, as well as using TEC buffers with CHARGESWITCH® beads.

FIG. 19A shows CHARGESWITCH® Size Selection Efficiency. An elution profile of a DNA fragment pool (35-1500 bp) bound to CHARGESWITCH® beads at low pH and eluted from the resin with buffers containing progressively more concentrated NaCl from 115-140 mM.

FIG. 19B shows a trace from on-chip electrophoresis (Agilent Bioanalyzer) of the samples from FIG. 19A to determine the fragment lengths eluted at each NaCl concentration.

FIG. 20A shows the effect of sodium dodecyl sulfate (SDS) on capture efficiency. A graph shows DNA recovered from TEC and CM beads without TEC ligands where the DNA was captured in the presence of varying concentrations of SDS.

FIG. 20B is a graph showing DNA recovered from TEC beads where the DNA was captured in the presence of PEG8000 and isopropyl alcohol under varying concentrations of SDS.

FIG. 21A is a graph showing the observed amount of captured DNA vs. the targeted amount returned from the three replicates of each normalization bead mixture. The inset shows the high correlation between the targeted amount of captured DNA and the observed amount returned from the experiment.

FIG. 21B is trace from on-chip electrophoresis (Agilent Bioanalyzer) of the samples from FIG. 21A to determine the fragment lengths eluted.

### DETAILED DESCRIPTION

This disclosure relates, in part, to methods and materials for recovering a target polyanion, such as a biomolecule, from a mixture using a specifically designed binding surface. The binding surfaces described herein comprise a solid support having tunable electrostatic capture (“TEC”) ligands bound to the surface of the solid support. In certain aspects, the TEC ligands comprise ionizable ligands having an ionizable functionality. In one embodiment, a linker covalently attaches the TEC ligand to a solid support. The ionizable functionality of the TEC ligand can be partially or fully electrostatically charged or neutral, depending on the pKa of the ionizable functionality and the pH of the medium or buffer in which the ionizable functionality is used or by which it is contacted.

The ionizable functionality of the TEC ligands can be adjusted to be positively charged when the pKa of the ionizable functionality is greater than pH of the binding conditions. Conversely, the ionizable functionality of the TEC ligands can be adjusted to be neutrally charged when the pKa of ionizable functionality is less than pH of the desorption or release conditions. In general, the binding and desorption or release conditions are dictated by the capture (binding) or release buffers, as described in more detail below.

In one embodiment, the target polyanion is a biomolecule. In one embodiment, the biomolecule is a nucleic acid, such as DNA or RNA. Because target biomolecules such as nucleic acids are negatively charged, TEC ligands are able to selectively bind or selectively elute or release the target biomolecules by adjusting the electrostatic charge of the TEC ligands, for example by altering the environment of the reaction mixture. Adjusting the ionizable functionality of the TEC ligands to be positively charged promotes binding of the negatively charged target biomolecule to the binding surface. Conversely, adjusting the ionizable functionality of the TEC ligands to be neutral or negatively charged inhibits binding of negatively charged target biomolecules to the binding surface or promotes release or elution of negatively charged target biomolecules bound to the binding surface.

Thus, the electrostatic nature of the ionizable functionality of the TEC ligands is “tunable,” and can be adjusted to either reversibly bind or release a desired target anion, such as a biomolecule, by adjusting the pH and/or ion (i.e., salt) concentrations of the binding and release conditions. This is in contrast to conventional anion exchange methods for the isolation of polyanions such as nucleic acids, wherein the charge of the resin generally does not change and where the system requires high salt concentrations to displace the bound nucleic acids.

Advantageously, the binding affinity of the TEC ligands can be further influenced without adjusting the electrostatic charge of the TEC ligands. For example, an increase in a concentration of ions in the medium in which the ionizable TE ligand functionality is used reduces the binding affinity of the TEC ligands for the target biomolecule, despite the ionizable functionality being positively charged at a pH that would otherwise enable binding of the target biomolecule to the binding surface (i.e., where the pKa of ionizable functionality is greater than pH of the binding conditions). Without wishing to be bound by theory, such an increase in the concentration of ions is believed to “screen” the positively charged ionizable TEC ligand functionality, thereby reducing its affinity for the target biomolecules. An increase in the concentration of ions can also screen a positively charged ionizable functionality that is bound to a target biomolecule. Thus, at a pH wherein the ionizable functionality is positively charged and bound to a target biomolecule, an increase in concentration of ions can screen the positive charge of the ionizable functionality to reduce its affinity for the target biomolecule, and promote release of the bound target biomolecule, all without any further adjustment in the pH of the reaction milieu.

As stated above, the ability to “screen” the electrostatic nature of the ionizable functionality by adjusting ion concentration while maintaining pH enables the binding affinity of the ionizable functionality to be further “tuned,” such that the ionizable functionality has reduced binding affinity for target biomolecules. The ion concentration can be adjusted to limit or reverse the binding of a target biomolecule to a positively charged, unbound ionizable functionality, or to limit the binding of a target biomolecule already bound to a positively charged ionizable functionality. For example, at a pH wherein the ionizable functionality is positively charged and would otherwise bind the target biomolecule to the binding surface (e.g., where the pKa of ionizable functionality is greater than pH of the binding conditions), an increase in the concentration of ions in the medium in which the ionizable functionality is used would reduce the binding affinity of the ionizable functionality for the target biomolecule, and inhibit binding of the target biomolecule. Likewise, at a pH wherein the ionizable functionality is positively charged and bound to a target biomolecule, an increase in the concentration of ions in the medium in which the ionizable functionality is used would reduce the binding affinity of the ionizable functionality for the target biomolecule, and promote release of the bound target biomolecule.

In one embodiment, “screening” the electrostatic nature of the ionizable functionality by increasing the ion concentration influences the binding affinity of the TEC ligands in a progressively size-selective manner. As smaller target polyanions, such as biomolecules, are generally less negatively charged than larger target polyanions, a progressive increase in the concentration of ions reduces the ability of the TEC ligands to bind target polyanions in a progressively size-selective manner (e.g., from smallest to largest).

Thus, variation of the pH of the binding conditions, along with precise electrostatic screening, provides a means by which polyanions such as nucleic acid molecules of different lengths can be selectively and/or sequentially retained or eluted from the TEC ligands. For example, in a sample solution containing only one type of nucleic acid, the method effectively produces a size selection, and allows for the precise “tuning” of the recovered molecule length. In one embodiment, in a complex sample mixture containing both RNA and DNA from a biological source, the size selection capability of TEC surfaces allows for generally rapid separation of RNA from DNA due to the relative size difference between the two with good efficiency.

Moreover, the size of the molecules that can be captured using TEC compositions is very low. For example, in one embodiment, nucleic acid molecules with only 35 bp or nucleotides, which is much smaller than the typical nucleic acid size standards for gel electrophoresis, have been captured using the present TEC compositions and methods. The low size limitation for nucleic acid capture is advantageous for the separation of very small nucleic acids, including micro RNAs, siRNAs, primers and small digested fragments of DNA. The described methods are capable of capturing and size-separating both single- and double-stranded nucleic acids, and it is understood that reference to nucleic acids herein, unless specifically mentioned or unless apparent from the context, encompasses both single- and double-stranded nucleic acids.

As discussed in more detail below, TEC ligands can be used in methods of separating polyanions from samples. In some embodiments, TEC ligands can be used in methods of separating target biomolecules from biological samples, clean-up or purification of nucleic acid samples, separating nucleic acids according to size, excluding nucleic acids according to size, and normalizing the concentration of nucleic acids from samples.

As used herein, “TEC functionality” or a “TEC ionizable functionality” refers to an ionizable functionality (i.e., a chemical moiety or structure) that allows the capture or binding of a target polyanion, such as a biomolecule, depending on the electrostatic nature of the ionizable functionality. In one embodiment, an ionizable functionality is an imidazole ring on a chemical compound. Ionizable ligands (i.e., the compound that comprise a TEC ionizable functionality) are referred to herein as “TEC ionizable ligands” or “TEC ligands”. In one embodiment, the compound histamine, as described in detail below, is the ionizable ligand having as its ionizable functionality an imidazole ring. Binding surfaces that comprise TEC ligands are referred to herein as “TEC binding surfaces” or “TEC surfaces”. In one embodiment, an TEC binding surface is the bead on which a histamine is covalently attached, such as the carboxy-modified magnetic beads obtained from Thermo Scientific on which the TEC ligand is bound. Example 1 provides one description of such a TEC binding surface. Methods using TEC functionalities, TEC ligands, or TEC surfaces are sometimes referred to therein as “TEC methods” or simply “TEC”.

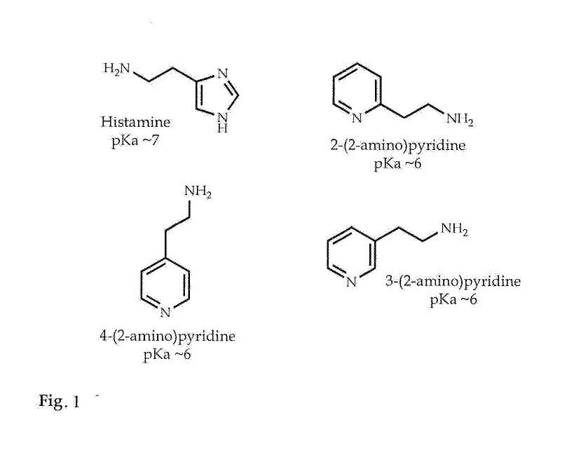

TEC ligands can be selected relative to the properties of the target polyanion, for example, a biomolecule, and can enable tunable electrostatic capture at or near biological pH. FIG. 1 shows several embodiments of TEC ligands that feature a primary amine and an aromatic amine moiety. The primary amine acts as a chemical ligation handle suitable for conjugation to a solid support. One embodiment of a TEC ligand depicted in FIG. 1 is histamine. The primary amine within histamine is suitable for conjugation to a solid support, while the imidazole side chain functions as a pH-dependent ionizable functionality. Imidazole has a pKa of 6.9 and thus at physiological pH, approximately half of the imidazole functional groups on the histamine side chain will be protonated. Lowering the pH of the environment to 5.9 will increase the percentage of protonated imidazole functional groups on the histamine side chain to approximately 90%, whereas increasing the pH to 7.9 will decrease the percentage of protonated imidazole functional groups on the histamine side chain to approximately 10%.

The ability to adjust or modulate the electrostatic nature of the TEC functionality by altering the pH of the buffer in this manner allows for one mode of selective control of the cationic nature of the TEC binding surface. As described in more detail below, a second mode for controlling the TEC binding surface is adjusting the ion concentration of the environment to “screen” the electrostatic nature of the ionizable functionality of the TEC ligand. Still other methods described herein rely on a combination and adjustment of both pH and ion concentration to manipulate binding and release of the polyanion targets to and from the TEC ligands and binding surface.

Other compounds can be selected for use as TEC ligands described herein based upon the properties of the target polyanion, for example a target biomolecule, and are not limited to histamine. Other suitable TEC ligands that meet the functional and structural requirements include but are not limited to 4-(2-amino)pyridine, 2-(2-amino)pyridine, 3-(2-amino)pyridine, imidazole, and pyridine or combinations thereof.

In embodiments where the TEC ligand is imidazole itself (which unlike histamine does not have the free primary amine moiety), a linker is required for attaching the ligand to the solid support. The “linker” refers to any moiety, generally a carbon chain used to attach or associate the TEC ligand to the solid support. In another embodiment the linker is composed of at least one to about 25 carbon atoms with a moiety such as a free amine group for conjugation. See, e.g., the descriptions of conjugation in the Examples 1-26 below. A linker, e.g., a carbon chain with a primary amine, may be attached to the imidazole ring at any suitable position. For example, the linker may be attached to the imidazole ring at the imidazole 4-position, the imidazole 2-position, or the imidazole 5-position.

In embodiments where the TEC ligand is pyridine, a linker for attaching the ligand to the solid support may be attached to the pyridine ring at any suitable position. For example, the linker may be attached to the pyridine ring at the pyridine 2-position, the pyridine 3-position, or the pyridine 4-position.

In one embodiment, TEC ligands are immobilized on a solid support. By the terms “support, solid support or surface” as used throughout this specification is meant a microparticle (bead), a microfluidics microparticle (bead), a slide, a multi-well plate or a chip. The supports or surfaces can be conventional, and include glass, plastic or any conventional materials suitable for the method or retrieval or selection of the desired polyanion. In some embodiments, the solid support is a bead, for example a magnetic bead. In another embodiment, the solid support can be a resin or collection of different types of resins or beads. In some embodiments, the solid support is a microfluidic chip. TEC ligands can also be immobilized on various other solid support systems. For example, in one embodiment, TEC nucleic acid capture can be accomplished on a microfluidic chip comprising immobilized TEC ligands. When combined with suitable devices for manipulating fluids containing biologic molecules or cells into and out from the microfluidic chip, the present methods enable the purification of nucleic acid molecules from micro volumes such as single cells, and further allows the subsequent analysis of the captured nucleic acid molecules with minimal dilution and generally in the absence of contaminants.

In one embodiment, the TEC ligands can be covalently attached to any solid support surface that is functionalized to undergo amine-reactive chemistry, including isothiocyanates, isocyanates, acyl azides, N-hydroxysuccinimide (NHS) esters and their corresponding sulfonated derivatives, sulfonyl chlorides, tosylate esters, aldehydes, epoxides, carbonates, aryl halides, imidoesters, anhydrides, fluorophenyl esters and hydroxymethyl phosphine derivatives.

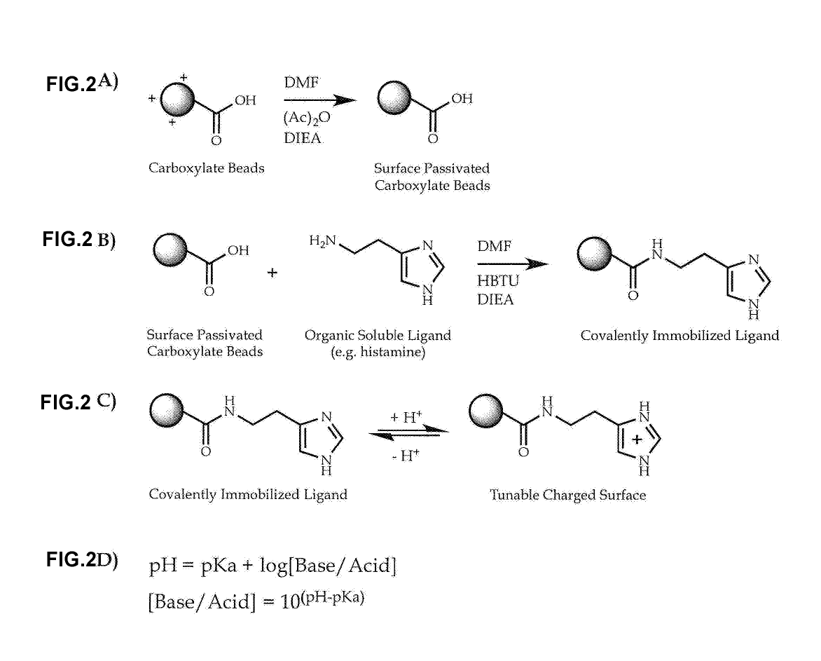

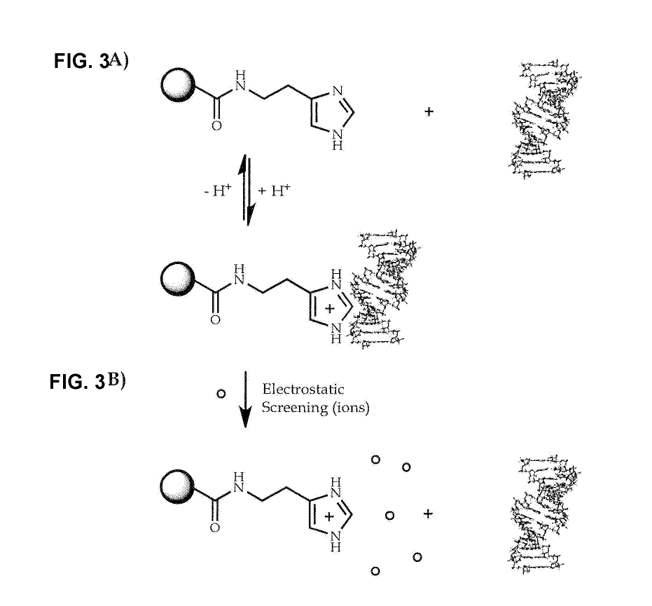

As shown in FIGS. 2A-2D, in one embodiment TEC ligands may be immobilized on the surface of magnetic carboxy-modified beads. Optionally, the carboxy-modified beads are treated to cap residual surface ionizable groups and to passivate the resin according to techniques known in the art. For example, TEC ligands can be immobilized on the surface of the carboxylate beads by conjugating TEC ligands to the carboxyl groups of the beads, using any suitable coupling chemistry known to one of skill in the art.

The extent to which a solid support is modified with TEC ionizable ligands can be varied. In general, a higher modification rate of the solid support surface with TEC ligands leads to a higher modification density, and produces a TEC binding surface that more tightly binds polyanions such as nucleic acids. Conversely, a lower modification rate of the solid support surface leads with TEC ligands to a lower modification density, and produces a TEC binding surface that binds polyanions such as nucleic acids less tightly.

The amount of TEC ligands bound to a given solid support surface is called the “modification density,” and can be expressed as a ratio of, for example, free carboxylic acid moieties on the solid support surface to TEC ligand bound to the solid support surface. In some embodiments, the ratio of free carboxylic acids to bound TEC ligand on a solid support surface is between about 1 to 9 and about 1 to 3. In some embodiments, the ratio of free carboxylic acids to bound TEC ligand is between about 1 to 3, or about 1 to 2, or about 1 to 1. In some embodiments, the ratio of free carboxylic acids to bound TEC ligand is about 1 to 3. In some embodiments, the ratio of free carboxylic acids to bound TEC ligand is about 1 to 1. In some embodiments, the ratio of free carboxylic acids to bound TEC ligand is about 3 to 1.

While a particular modification density may be ideal for certain applications, the modification density can be increased or decreased to “fine tune” the TEC binding surface for the capture of various polyanions, such as various of nucleic acids. For example, a lower modification density can be achieved through sub-quantitative coupling of the TEC ligand to the solid support. In one embodiment, sub-quantitative coupling of histamine to a solid support containing carboxylic acid functional groups will yield a TEC binding surface with both histamine functional groups and free carboxylic acid functional groups, the latter of which will be deprotonated at physiological pH. The resulting negatively charged carboxylate functional groups may help prevent non-specific interactions of polyanions with the TEC-derivatized solid support. Sub-quantitative coupling of histamine can be accomplished through any suitable chemistry known to one of skill in the art, including (1) sub-quantitatively adding a carbodiimide/uranium coupling reagent and coupling with an excess of histamine, or (2) co-coupling histamine with an amino acid in various ratios.

As noted above, the TEC ligands are positively charged when the pKa of the ionizable functionality is greater than pH of binding conditions. Conversely, the ionizable functionality of the TEC ligands can be adjusted to be neutrally charged when the pKa of ionizable functionality is less than pH of the desorption or release conditions. Thus, contacting the TEC-derivatized solid support with a TEC capture buffer having a pH less than the pKa of the ionizable ligand causes the TEC ligands to have a positive charge. The pH that causes the TEC ligands to have a positive charge will depend on the identity of the TEC ligand, but is suitably less than about 8, for example less than about 7, or less than about 6.

Polyanions are defined as biological or chemical compounds or compositions having negative charges at one or multiple sites. In one embodiment, polyanions suitable for capture and/or size selection according to the methods described herein, include nucleic acids that can be isolated from various biological samples.

A biological sample can be a naturally-occurring sample or deliberately designed or synthesized sample or library containing polyanions. In one embodiment, the sample contains a population of cells or cell fragments, including without limitation cell membrane components, exosomes, and sub-cellular components. The cells may be a homogenous population of cells, such as isolated cells of a particular type, or a mixture of different cell types, such as from a biological fluid or tissue of a human or mammalian or other species subject. The biological sample can be simple, for example containing isolated DNA or RNA or deriving from a homogeneous cell culture or tissue source, or can be complex, such as deriving from tumor, blood or whole organ samples. The biological sample can be from any suitable source, such as a healthy tissue or cell source, a diseased tissue or cell source, a cell culture or line, cell extracts or lysates, a biopsy, and the like.

Still other samples for use in the methods and with the kits include, without limitation, blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal or rectal). Still other samples include a blood-derived or biopsy-derived biological sample of tissue or a cell lysate (i.e., a mixture derived from tissue and/or cells). Other suitable tissue includes hair, fingernails and the like. Still other samples include libraries of antibodies, antibody fragments and antibody mimetics like affibodies. Such samples may further be diluted with saline, buffer or a physiologically acceptable diluent. Alternatively, such samples are concentrated by conventional means. Still other samples can be synthesized or engineered collections of chemical molecules, proteins, antibodies or any other of the polyanions described herein.

Polyanions, such as nucleic acids can be extracted from any suitable living source, including human, animal, microbial, plant or viral sources, including cells, saliva, fresh tissue or other materials containing genetic DNA or RNA, whether initially whole or otherwise wholly or partially disrupted. Prior to contacting the biological sample to a solid support covalently bound to a TEC ligand, the sample can be disrupted, disaggregated, homogenized, or lysed by any technique known in the art suitable for liberating the nucleic acids from the sample. For example, the biological sample may be made into a single-cell suspension using a nylon filter or mesh. The cells can then be washed and then lysed according to techniques known to one skilled in the art.

Binding of polyanions, such as nucleic acids in a biological sample, to the TEC ligands is carried out by contacting the sample with a solid support covalently bound to TEC ligands under conditions which allow the TEC ligand to bind the polyanion. Buffers having pH and ionic conditions under which polyanions, for example from biological samples, bind to TEC ligands are referred to herein as “TEC-capture buffers.”

Binding of nucleic acids from biological samples generally occurs in a buffer having a pH that is below 7 and which is below the pKa of the TEC functionality or TEC ligand. As certain desirable polyanion targets have pKA of between about 6 to 8, in these embodiments, the pH at which the solid support bound TEC ligand binds the nucleic acids is between about 4.0 and about 5.5. Therefore, the TEC capture buffer has a pH of between 4.0 and 5.5. In some embodiments, the pH at which the solid support bound TEC ligand binds the nucleic acids is about 4.0. In some embodiments, the pH at which the solid support bound TEC ligand binds the nucleic acids is about 4.5. In some embodiments, the pH at which the solid support bound TEC ligand binds the nucleic acids is about 4.8. Examples of suitable TEC-capture buffers for binding nucleic acids to a TEC ligand include sodium acetate, sodium acetate with Tween-20, sodium acetate with sodium chloride, and sodium acetate with sodium chloride and Tween-20. Specific examples of TEC capture buffers are described below in the examples, and referred to as binding buffers or capture buffers. In one embodiment, Example 1 described a 10× capture buffer containing 500 mM sodium acetate, pH 4.0 with 0.1% Tween-20. Example 4 describes several capture buffers as 10×pH 4.8 binding buffer: 1 M NaOAc (Ambion), 1 M NaCl (Ambion), 0.1% Tween-20 (Enzo Life Sciences), pH 4.8; and 10×pH 4.0 binding buffer: 1 M NaOAc (Ambion), 1 M NaCl (Ambion), 0.1% Tween-20 (Enzo Life Sciences), pH 4.0. A number of different TEC capture buffers are defined in detail in the remaining examples below.

Binding of nucleic acids from biological samples generally occurs in a buffer having an ion (salt) concentration of less than about 1000 mM. In one embodiment, the salt concentration of the buffer during binding is between about 50 mM and about 700 mM. In some embodiments, the ion concentration is at least 60, 75, 100, 150, 200, 250, 300, 350, 400, 450, 500, 550, 600, 650 or up to 700 mM. A suitable salt for use in TEC capture buffers is sodium chloride, although other salts may be used without limitation.

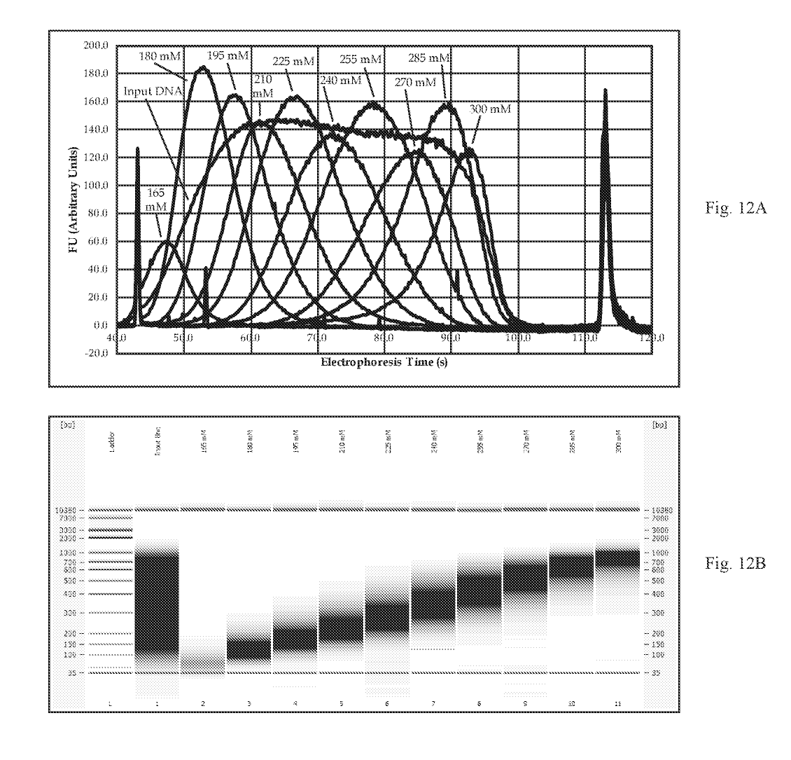

If desired, it is possible in the present methods to inhibit nucleic acids having a size lower than a desired threshold (such as oligonucleotide primers or damaged or degraded nucleic acid fragments) from binding to TEC ligands. By inhibiting the initial binding of nucleic acids having a size lower than a desired threshold, size exclusion methods can advantageously purify nucleic acids having a desired minimum size from a biological sample by a single step. Size exclusion may be accomplished by providing a TEC—capture buffer having a salt concentration configured to inhibit binding of nucleic acids having a size lower than the desired threshold, but which allow binding of nucleic acids having a size greater than the threshold. When performing size exclusion, methods according to the invention, the salt concentration of the TEC-capture buffer may be selected depending on the size of the nucleic acid that is desired to be excluded. See, for example FIGS. 12A and 12B.

In some embodiments of methods of the invention, the nucleic acid bound TEC ligand is optionally washed to remove impurities and unbound nucleic acids. The choice of pH for washing of the nucleic acid bound TEC ligand will generally depend on the identity of the TEC ligand used to bind the nucleic acid. For example, washing of the nucleic acid bound TEC ligand can occur at a pH that is below 6.6, or at any suitable pH wherein the nucleic acid remains substantially bound to the TEC ligand or wherein the nucleic acid is substantially not released from the TEC ligand. The nucleic acid bound TEC ligand can also be washed at a pH which is the same or substantially the same the pH of binding.

In some embodiments, the pH at which the nucleic acid bound TEC ligand is washed is about 6.5. In some embodiments, the pH at which the nucleic acid bound TEC ligand is washed is about 6.0. In some embodiments, the pH at which the nucleic acid bound TEC ligand is washed is about 5.5. In some embodiments, the pH at which the nucleic acid bound TEC ligand is washed is about 5.3. In some embodiments, the pH at which the nucleic acid bound TEC ligand is washed is about 4.8.

Examples of suitable buffers for washing nucleic acid bound TEC ligands (also known as TEC release buffers) include sodium phosphate, sodium phosphate with Tween-20, sodium acetate, and sodium acetate with Tween-20. In some embodiments, the buffer used to wash the nucleic acid bound TEC ligand comprises a 10 mM sodium phosphate solution and Tween-20, wherein the pH of the buffer is about 6.0. In some embodiments, the buffer used to wash the nucleic acid bound TEC ligand comprises a 10 mM sodium acetate solution and Tween-20, wherein the pH of the buffer is about 6.0. In one embodiment described in Example 4, a wash or release buffer is 10 mM sodium phosphate (Amresco), 0.01% Tween-20 (Enzo Life Sciences), pH 6.0.

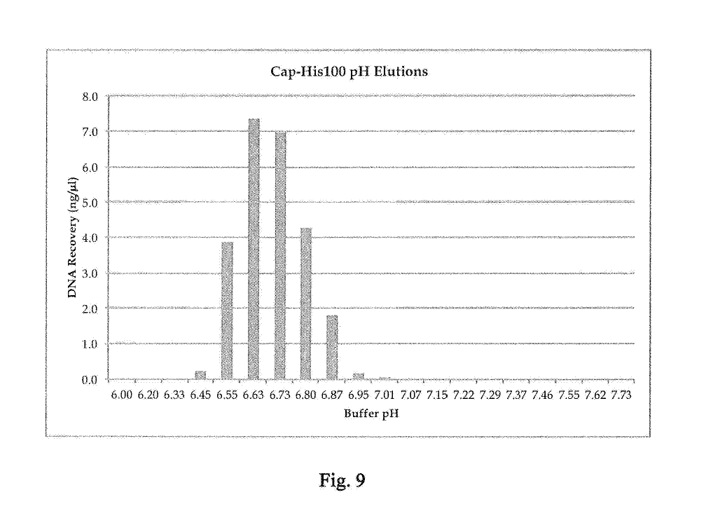

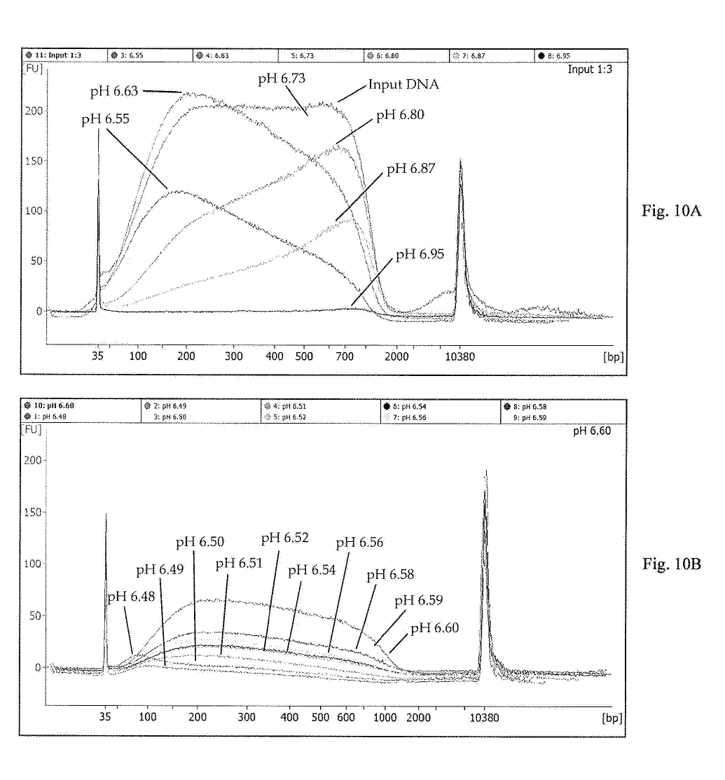

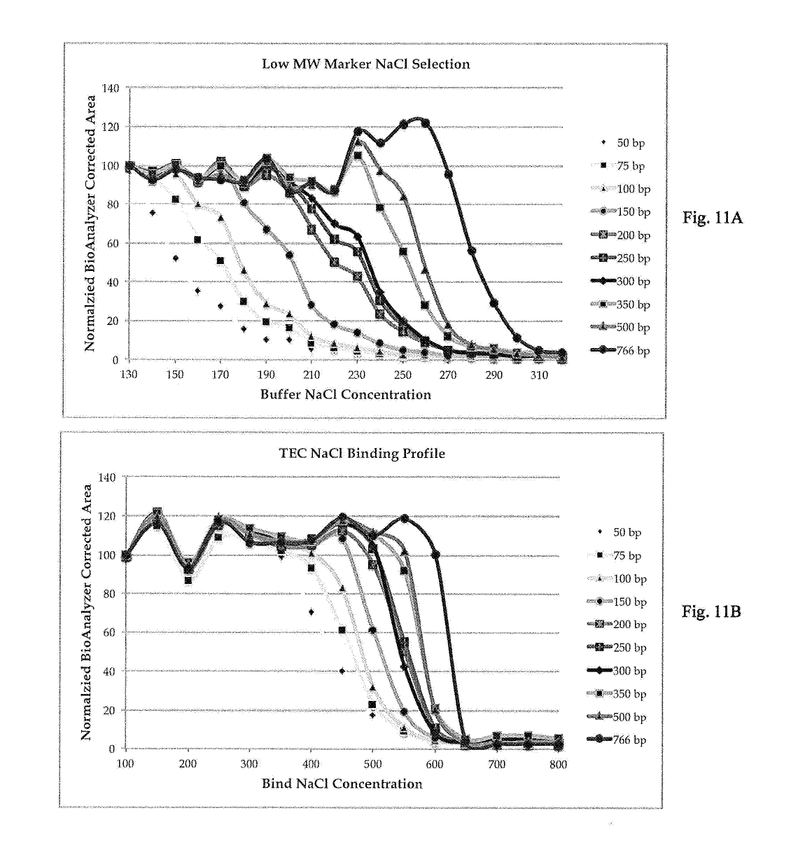

In certain embodiments of the methods described herein, nucleic acids bound to the TEC ligands can be released by reducing the binding affinity of the TEC ligands to the nucleic acids. As noted above, changes in buffer pH and buffer ionic strength can both be used to adjust or “tune” the binding affinity of nucleic acid bound TEC ligands, such that the bound nucleic acids are released. It was surprisingly found that changes in buffer pH without changing the buffer ionic strength does not meaningfully release nucleic acids bound to TEC ligands in a size selective manner, as is shown for example in FIGS. 9 and 10. In other embodiments, changes in buffer pH without changing the buffer ionic strength can still be suitably used in these methods to perform DNA clean-up, and to separate nucleic acids from impurities or release nucleic acids in a concentration-dependent manner. Conversely, changes in buffer ionic strength can be used adjust the binding affinity of nucleic acid bound TEC ligands, such that the bound nucleic acids are released in a size selective manner, as shown for example in FIGS. 12A and 12B and 13.

In either case, buffers having pH and/or ionic conditions under which polyanions from biological samples bound to TEC ligands are released from the TEC ligands are referred to herein as “TEC-release buffers.” Examples of suitable TEC-release buffers for releasing a nucleic acid which is bound to a TEC ligand include tris(hydroxymethyl)aminomethane (Tris), tris(hydroxymethyl)aminomethane with sodium chloride, tris(hydroxymethyl)aminomethane with sodium chloride and Tween-20, tris(hydroxymethyl)aminomethane with Tween-20, and imidazole. In some embodiments, the TEC-release buffer used to release a nucleic acid which is bound to a TEC ligand comprises a 10 mM sodium phosphate solution and Tween-20, wherein the pH of the buffer is less than about 7.0. In some embodiments, the buffer used to release a nucleic acid which is bound to a TEC ligand comprises a sodium chloride solution, wherein the pH of the buffer is about 6.0. In some embodiments, the buffer used to release a nucleic acid which is bound to a TEC ligand is an imidazole solution, wherein the pH of the buffer is about 6.0. In other embodiments the release buffers have a pH of between 5.0 and 6.5, such as 5.2, 5.5, 5.7, 6.0, 6.2, 6.3 and 6.5.

In some embodiments, releasing a nucleic acid which is bound to a TEC ligand by changing the pH may occur at a pH below about 8.5. As discussed above, the choice of pH for releasing a nucleic acid which is bound to a TEC ligand will depend on the identity of the TEC ligand, the conditions required of the step or steps following nucleic acid release and, to a lesser extent, on the size of the nucleic acid to be isolated. In some embodiments, the nucleic acid is released from the TEC ligand at a pH between about 7.5 and about 8.3. In some embodiments, the nucleic acid is released from the TEC ligand at a pH between about 6.5 and about 7.5. In some embodiments, the nucleic acid is released from the TEC ligand at a pH between about 6.0 and about 7.0. In some embodiments, the nucleic acid is released from the TEC ligand at a pH between about 6.0 and about 6.9. In some embodiments, the nucleic acid is released from the TEC ligand at a pH between about 5.5 and about 6.5. In some embodiments, the nucleic acid is released from the TEC ligand at a pH between about 5.0 and about 6.0. In some embodiments, the pH of the buffer used to release the nucleic acid is about the same as the pH of the buffer used to wash the nucleic acid bound TEC ligand, but the ionic strength of the buffer used to release the nucleic acid is greater than the ionic strength of the buffer used to wash the nucleic acid bound TEC ligand.

For elution buffers, in which the goal is to remove all bound nucleic acid regardless of size, the buffer may have a pH greater than the pKa of the TEC ligand. In certain circumstances, the elution buffer has a pH greater than 8, 8.5, 9.0, or 9.5 or fractional numbers therebetween.

Releasing a nucleic acid which is bound to a TEC ligand by changing the buffer ionic strength can be accomplished by contacting the nucleic acids bound to TEC ligands with a buffer having a progressively increasing a salt concentration. This may be accomplished, for example, by adding a salt to the liquid medium containing the solid support with TEC ligands bound to nucleic acids, or by transferring the solid support with TEC ligands bound to nucleic acids to one or more different liquid mediums having progressively higher salt concentrations. Generally, the salt concentration of the TEC-release buffers is about 1000 mM or less. In one embodiment, the salt concentration of the buffer during binding is between about 50 mM and about 700 mM.

In one embodiment, nucleic acids bound to TEC ligands may be released from the TEC ligands according to size, by contacting the solid support to which the TEC ligands are bound with at least a first buffer having a first salt concentration. This causes nucleic acids having a first size to be released from the TEC ligands, while nucleic acids having the second size greater than the first size remain bound to the TEC ligands. If further separation by size of the nucleic acids remaining bound to the TEC ligands is desired, the solid support with TEC ligands bound to nucleic acids having the second size may be contacted with at least a second buffer having a second salt concentration greater than the first salt concentration. This releases the nucleic acids having a second size from the TEC ligands. In some examples, nucleic acids having a third or additional size greater than both the first size and the second size remain bound to the TEC ligands after contacting the solid support with the second buffer. Optionally, the solid support with the bound TEC ligands may be contacted with a third buffer or yet further additional buffers having progressively greater ionic strength or salt concentration, to continue to separate nucleic acids according to size. There is no limit on the number of buffers having different ionic strengths which can be successively employed in size-separation methods of the invention. For an example of size release by such TEC release buffers, see FIGS. 12A and 12B.

When releasing nucleic acids in a size selective manner according to the present methods, the concentration, for example, the molar concentration, can be progressively increased in each separation step. In one embodiment, the progressive increase in salt concentration is effected by each successive buffer used. In some embodiments, the molar concentration of salt in the TEC-release buffers is, for example, progressively increased by at least about 10 mmole/L for each step of the size-selection. In some embodiments, the molar concentration of salt in the TEC-release buffers is, for example, progressively increased by at least about 15 mmole/L for each step of the size-selection. In some embodiments, the molar concentration of salt in the TEC-release buffers is, for example, progressively increased by at least about 25 mmole/L for each step of the size-selection. In some embodiments, the molar concentration of salt in the TEC-release buffers is, for example, progressively increased by at least about 35 mmole/L or more for each step of the size-selection.

In one embodiment, the invention provides a method of purifying nucleic acids in preparation for further biological investigations, including nucleic acid sequencing, such as next-generation sequencing applications. For next-generation sequencing applications, TEC ligands can be attached to the surface of magnetic beads, yielding TEC magnetic beads that are an improvement over the current solid phase reversible immobilization (SPRI) bead technology. Compared to SPRI, TEC offers a rapid and efficient method for isolating and size separating nucleic acids. For example, TEC methods of the invention can typically be carried out within about 3 minutes, as opposed to significantly longer times (i.e, on the order of tens of minutes to hours) for SPRI. The volumes used in TEC are also smaller compared to the volumes used in SPRI. Furthermore, unlike SPRI, TEC does not require the use of polyethylene glycol, ethanol, or solutions with high salt concentration, which can result in longer processing times in order to ultimately rid the sample of these materials. As discussed above and as shown in the Examples below, the methods described herein are broadly “tunable,” flexible and can be adapted to many different applications. Such applications include reaction clean-up for DNA and RNA, small nucleotide capture, DNA size-selection, complex purification using tandem binding resins with different binding profiles, genomic DNA and RNA capture from tissue/cells, fragmented DNA/RNA capture from FFPE (formalin-fixed, paraffin-embedded) material, normalization of nucleic acid concentration across a plurality of samples, and total nucleic acid separation.

In one embodiment, TEC ligands may be used in a method for the capture of fragmented nucleic acids, such as DNA and/or RNA from FFPE material. Compared, for example, to the Covaris truXTRAC extraction technology, the method described herein is faster, recovers more nucleic acids, and recovers higher quality nucleic acids (as measured e.g., by the length distribution of the recovered nucleic acids). Generally, there are four main steps in capturing nucleic acidsfrom FFPE: 1) Paraffin removal, 2) tissue digestion, 3) formaldehyde cross link reversal, and 4) nucleic acidrecovery. Paraffin removal is typically done with organics (xylenes, etc.) and is not high throughput, typically requiring some time to complete. Tissue digestion is typically performed with a proteinase such as proteinase K (which operates at 55° C.), and this step can require 1-3 hours or more. The step of reversing formaldehyde cross links is dependent on time and temperature, with the optimum conditions usually comprising incubating the FFPE material at 75-80° C. for approximately one hour. Nucleic acid recovery is traditionally performed with silica spin columns and chaotropic salts (e.g. Guanidinium), which can also be time consuming.

In contrast, one embodiment of the present method combines the first three steps (paraffin removal, tissue digestion, and cross link reversal) into a single step which can be performed in one tube. Rather than using deparaffinizing reagents, heat is used to melt the plastic. For tissue digestion, a heat-stable protease (for example from Kapa Biosystems), which is active between about 75° C. and about 80° C., is used. Thus the steps of paraffin removal, tissue digestion, and cross link reversals can be performed at the same time without any additional handling steps in between, providing a faster method. Nucleic acids which have been liberated from the FFPE material are then isolated using TEC surfaces (e.g., TEC-derivatized magnetic beads), which is efficient, fast, and amendable to automation. The overall process for capture of fragmented nucleic acids from FFPE material is thus faster and more efficient.

Other variations to the present method will be apparent to one skilled in the art. For example, in some embodiments, the heating step for tissue digestion is performed at about 75° C. In some embodiments, the heating step for tissue digestion is performed at about 80° C. In some embodiments, the heating step for tissue digestion is followed by a cooling step. In some embodiments, the heating step for tissue digestion is followed by a cooling step and a filtration step. In some embodiments, the heating step for tissue digestion is followed by a filtration step. In some embodiments, the liberated nucleic acids are isolated using Cap-His100 TEC beads. In some embodiments, the liberated nucleic acids are isolated using TEC beads at room temperature. In some embodiments, the liberated nucleic acids are isolated using TEC beads at ambient temperature. In some embodiments, the liberated nucleic acids are isolated using TEC beads with heating. In some embodiments, the liberated nucleic acids are isolated using TEC beads at a temperature between about 42° C. and about 55° C.

In other embodiments, TEC ligands may be used in a method for normalizing the concentration of polyanions, such as nucleic acids, among a plurality of samples. The ability to rapidly and efficiently obtain a normalized concentration of nucleic acids across a plurality of samples is advantageous because it precludes the need to perform additional steps to standardize the DNA concentration for later sequencing applications. In one embodiment, normalization methods of the invention comprise obtaining a normalized concentration of nucleic acids from a plurality of samples, which normalized concentration in a given volume comprises the amount of nucleic acid required for direct loading into an automated nucleic acid sequencer.

A method for normalizing a concentration of nucleic acids from a plurality of samples according to the invention can comprise providing a plurality of biologic samples comprising nucleic acids, wherein at least one of the plurality of biologic samples has a different concentration of nucleic acid than the other samples. For example, all or substantially all of the biologic samples can have different nucleic acid concentrations. A solid support covalently bound to TEC ligands is prepared as described herein, and each biologic sample is mixed with a fixed and substantially similar amount of this solid support in liquid medium under substantially the same conditions, in which the mixture has a pH less than the pKa of the ionizable ligand. This allows the TEC-ligands to reversibly bind substantially the same amount of nucleic acids from each biologic sample. The solid support covalently bound to TEC ligands is then recovered from each biologic sample according to the techniques described herein, and kept separate. For example, if the solid support comprises a bead, then it can be isolated from the biologic sample by centrifugation. If the solid support comprises a magnetic bead, then it can be isolated from the biologic sample by magnetic separation. The recovered solid support can optionally be subjected to one or more washing steps as described herein, and/or can be subjected to one or more steps which remove any residual liquid. The skilled person can readily determine, based on the description provided herein, what steps may be desirable or necessary to recover the solid support and prepare it for releasing the bound nucleic acids. A substantially similar amount of nucleic acids can then be released from the TEC-ligands bound to the solid support recovered from each biologic sample (provided a sufficient nucleic acid concentration was initially present in a given sample), by contacting the solid support with one or more buffers having a pH greater than the pKa of the ionizable ligand, according to the techniques described herein. For example, substantially all of the bound nucleic acids can released at once, or the solid support covalently bound to TEC ligands can be subjected to two, three or more buffers designed to release the bound nucleic acids in a size-selective manner. In one embodiment, the bound nucleic acids are released substantially simultaneously.

The nucleic acids thus released from the solid support covalently bound to TEC ligands recovered from each biologic sample can be reconstituted or diluted in a substantially similar amount of vehicle (such as a storage or sequencing buffer), thus resulting in a “normalized” (i.e., substantially similar) concentration of nucleic acid recovered from each biologic sample. For example, in one embodiment the released nucleic acids can be precipitated and dried for reconstitution later in a fixed amount of a liquid vehicle. In another embodiment, the released nucleic acids can remain in the elution buffer without further concentration or dilution. In yet another embodiment, the released nucleic acids can be diluted in or from the elution buffer up to a predetermined concentration.

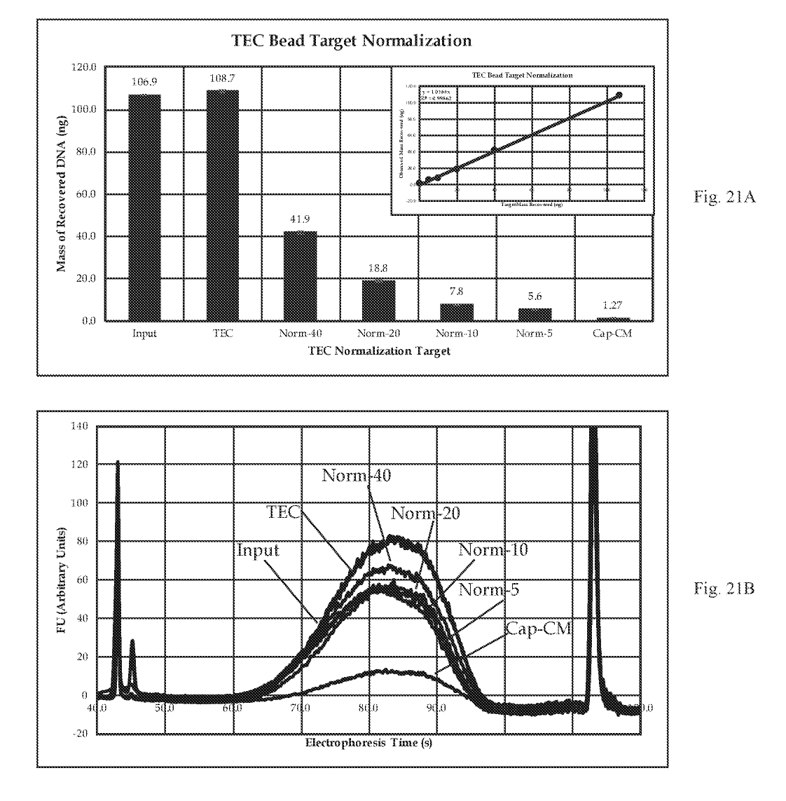

In normalization methods of the invention, the amount of nucleic acid bound and then released from each of the plurality of biologic samples can be predetermined. For example, if the amount of nucleic acid needed for a particular procedure (e.g., sequencing) is known, then an amount of solid support covalently bound to TEC ligands which is expected to, or has been previously demonstrated to, bind that amount of nucleic acid can be added to each biologic sample. Because the normalization methods of the invention quickly, efficiently and reliably capture substantially similar amounts of nucleic acids from biologic samples, even from biologic samples with widely different overall amounts of nucleic acid, there is typically no need to quantitate the recovered nucleic acid before proceeding to use it in sequencing or other procedures. However, one may include one or more quantitation steps if desired. An exemplary normalization method of the invention is shown in Example 20 below. As shown in Example 20 and FIGS. 21A and 21B, recovery of nucleic acids by normalization methods of the invention shows linearity over a very large range, even down to unmeasurable amounts of nucleic acid.

In yet another aspect, kits are provided for performance of one or more of the methods described herein. In one embodiment, a solid support comprising a TEC ligand may be provided in a kit optionally in combination with one or more TEC-capture buffers and TEC-release buffers, and optionally with instructions for performing the methods of the invention. For example, the instructions may provide details on making, storing and using the TEC-capture and/or TEC-release buffers. A kit of the invention may comprise a solid support which is not bound to any TEC ligands, and one or more TEC ligands, with reagents and optionally instructions suitable for covalently binding the solid support to the TEC ligands. A kit of the invention may also comprise at least a solid support covalently bound to a multitude of TEC ligands, at least one TEC-capture buffer having a pH less than the pKa of the ionizable ligand, and at least one TEC-release buffer. Optionally, a kit may include a first TEC-release buffer for releasing nucleic acids having a first size, the first TEC-release buffer having a salt concentration, and a second TEC-release buffer for releasing nucleic acids having a second size greater than the first size, the second TEC-release buffer having a salt concentration greater than the salt concentration of the first TEC-release buffer. Additional TEC-release buffers may be provided as desired.

The TEC system and kits containing the components of the systems may be utilized to accomplish a variety of research tasks, including, without limitation, nucleic acid (DNA and RNA) cleanup (PCR, enzyme reactions, etc), small RNA (miRNA) purification, fractionation of miRNA from mRNA, nucleic acid size selection (primer removal from PCR, etc), and NGS library prep (cleanup & size selection). The methods and kits described herein are also useful in nucleic acid extraction (DNA and RNA) from, for example, FFPE, fresh frozen tissue, stabilized tissue, circulating cell-free DNA (cfDNA), environmental samples, and other tissue/fluid types (saliva, stool, etc).

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent higher or lower.

The terms “first”, “second” and “additional” are used throughout this specification as reference terms to distinguish between various components of methods. As one example, the “first size” of the target polyanion nucleic acid fragment is meant to distinguish that size from a “second”, i.e., differently sized fragment or from any “additional” fragment.

The terms “a” or “an” refers to one or more. For example, “a TEC release buffer” is understood to represent one or more such buffers. As such, the terms “a” (or “an”), “one or more,” and “at least one” are used interchangeably herein.

The words “comprise”, “comprises”, and “comprising” are to be interpreted inclusively rather than exclusively, i.e., to include other unspecified components or process steps.

The words “consist”, “consisting”, and its variants, are to be interpreted exclusively, rather than inclusively, i.e., to exclude components or steps not specifically recited.

As used herein, the phrase “consisting essentially of” limits the scope of a described composition or method to the specified materials or steps and those that do not materially affect the basic and novel characteristics of the described or claimed method or composition. Wherever in this specification, a method or kit is described as “comprising” certain steps or features, it is also meant to encompass the same method or composition consisting essentially of those steps or features and consisting of those steps or features.

In the descriptions of the kits, compositions, components and methods discussed herein, the various components can be defined by use of technical and scientific terms having the same meaning as commonly understood by one of ordinary skill in the art to which this invention belongs and by reference to published texts. Such texts provide one skilled in the art with a general guide to many of the terms used in the present application. The definitions contained in this specification are provided for clarity in describing the components and compositions herein and are not intended to limit the claimed invention.

It will be recognized that one or more features of any embodiments disclosed herein may be combined and/or rearranged within the scope of the invention to produce further embodiments that are also within the scope of the invention. Those skilled in the art will recognize, or be able to ascertain using no more than routine experimentation, many equivalents to the specific embodiments of the invention described herein. Such equivalents are intended to be within the scope of the present invention.

### Embodiments

Specific embodiments of the methods and compositions described herein are listed below:

In one embodiment a method for separating polyanions according to size from a sample comprises: contacting a sample which comprises polyanions of different sizes with a solid support to which is covalently bound a plurality of tunable electrostatic capture (TEC) ligands. The TEC ligands comprise an ionizable ligand which is positively charged at a pH less than a pKa of the ionizable ligand and neutral at a pH greater than the pKa of the ionizable ligand. The support was previously contacted with a TEC capture buffer having a pH less than the pKa of the ionizable ligand, which causes the TEC ligands to have a positive charge. In this method, the sample is contacted with the solid support under conditions which allow the TEC ligands to reversibly bind polyanions of different sizes in the sample to the TEC ligands having a positive charge. The method additionally includes a step of releasing the polyanions from the TEC ligands according to size by contacting the solid support with at least a first TEC release buffer having an ion concentration suitable for releasing polyanions having a first size from the TEC ligands. After the solid support which has bound thereto polyanions from the sample is contacted with the first TEC release buffer, polyanions having a second size greater than the first size remain bound to the TEC ligands.

In another embodiment, the above method can include the steps of providing a plurality of tunable electrostatic capture (“TEC”) ligands covalently bound to a solid support, wherein the TEC ligands comprise an ionizable ligand which is positively charged at a pH less than a pKa of the ionizable ligand and neutral at a pH greater than the pKa of the ionizable ligand. The method can also include the active step of causing the TEC ligands to have a positive charge by contacting the solid support with a TEC capture buffer having a pH less than the pKa of the ionizable ligand.

In either embodiment described above, the methods can further comprise contacting the solid support with at least a second TEC release buffer having a second ion concentration greater than the ion concentration of the first TEC release buffer. By performance of this step, the polyanions having a second size greater than the first size are released from the TEC ligands.

In yet a further embodiment, the methods described above can further comprising contacting the solid support with at least one additional TEC release buffer having an additional ion concentration greater than the ion concentration of the second, wherein the polyanions having a size greater than the second size are released from the TEC ligands. In another embodiment, where a first, second and multiple additional TEC release buffers are sequentially applied to the solid support, each additional TEC release buffer permits release of progressively larger poly anions from the TEC ligands, because each additional TEC release buffer has a ion concentration greater than of its preceding additional TEC release buffer. Any number of TEC release buffers can be employed in the method depending upon the number of differently sized polyanions that are present in the sample.

In yet a further embodiment of the methods described above, each method comprises actives steps of separating, isolating and collecting from the sample the polyanions of more than one size that are released by the successive TEC release buffers.

Various embodiments of the methods involve wherein the sample is a sample of biological components and polyanions, wherein the polyanions comprise nucleic acids and/or wherein the nucleic acids are DNA or RNA. Still other embodiments involve performing any of the methods, wherein the pH of the liquid medium or TEC capture buffer is less than about 7 or wherein the pH of the liquid medium or TEC capture buffer is between about 4.0 and about 5.0, or wherein the pH of the TEC capture buffer is pH 4, 5, 6 or 7 or fractional numbers therebetween.

Still other embodiments of the methods involve using TEC-release buffers having a pH of less than about 7. In certain embodiments, the TEC-release buffers have a pH of about 7, 6, 5, 4, 3, 2 or 1, including fractional numbers therebetween. In some embodiments of the method, the ion concentration of each TEC release buffer is about 1000 mmole/L or less. In other embodiments of the methods, a molar concentration of ion in the first TEC release buffer is at least about 10 mmole/L less than a molar concentration of ion in the second TEC release buffer. In still other embodiments of the method, a molar concentration of ion in the second TEC release buffer is at least about 10 mmole/L less than a molar concentration of ion in a third or additional TEC release buffer.

Other aspects of the methods involve the ionizable ligand being a heterocyclic amine. In still other embodiments, the ionizable ligand used in the method is histamine. In still other embodiments, the ionizable ligand used in the method is 4-(2-amino)pyridine, 3-(2-amino)pyridine, 2-(2-amino)pyridine, pyridine or imidazole.

Yet other embodiments of the methods employ as the solid support a bead, a magnetic bead, or a microfluidic chip. In some embodiments, the solid support further comprises free carboxylic acid functional groups. In some embodiments, the ratio of free carboxylic acids to TEC ligand bound to the solid support is between about 1 to 9 and about 1 to 3. In other embodiments, the ratio of free carboxylic acids to TEC ligand bound to the solid support is between about 1 to 3 and about 1 to 1.

Embodiments of the kit are provided herein including a kit comprising a plurality of tunable electrostatic capture (“TEC”) ligands covalently bound to a solid support, wherein the TEC ligands comprise an ionizable ligand which is positively charged at a pH less than the pKa of the ionizable ligand and neutral at a pH greater than the pKa of the ionizable ligand. The kit also contains a TEC capture buffer having a pH less than the pKa of the ionizable ligand. The kit also contains a set of TEC buffers. Among the set of buffers includes a TEC wash buffer having a pH greater than that of the capture buffer but less than that of the ionizable ligand on the TEC support. The kit also includes in certain embodiments, a first TEC release buffer suitable for releasing polyanions having a first size, the first TEC release buffer having a first ion concentration. In certain embodiments, the kit includes a second TEC release buffer suitable for releasing polyanions having a size greater than the first size, the second TEC release buffer having an ion concentration greater than the ion concentration of the first TEC release buffer. Included in the set of buffers is also, a TEC elution buffer having a pH greater than that of the ionizable ligand on the TEC support.

In yet other embodiments, the kit comprises at least one additional TEC release buffer suitable for releasing polyanions having an additional size greater than the second size, each said additional buffer having an ion concentration greater than the ion concentration of the second TEC release buffer and different from the ion concentration of any other additional TEC release buffer in the kit.

In still another embodiment a kit for separating polyanions according to size from a sample comprising polyanions of different sizes, comprises a plurality of tunable electrostatic capture (“TEC”) ligands covalently bound to a solid support, wherein the TEC ligands comprise an ionizable ligand which is positively charged at a pH less than a pKa of the ionizable ligand and neutrally charged at pH greater than the pKa of the ionizable ligand. The kit also contains a TEC-capture buffer having a pH less than the pKa of the ionizable ligand; and a set of TEC-release buffers. This set comprises at least a first TEC-release buffer for releasing polyanions having a first size, the first TEC-release buffer having a salt concentration, and at least a second TEC-release buffer for releasing polyanions having a second size greater than the first size, the second TEC-release buffer having a salt concentration greater than the salt concentration of the first TEC-release buffer. In other embodiments, such a kit further comprises at least a third TEC-release buffer having a salt concentration greater than the salt concentration of the second TEC-release buffer, wherein the polyanions having a third size greater than the second size are released from the TEC ligands.

In various embodiments, these kits are designed for use with a sample of biological components and polyanions, particularly wherein the polyanions comprise nucleic acids and/or wherein the nucleic acids are DNA or RNA.

In other embodiment, the liquid medium or TEC capture buffer in the kits has a pH less than about 7 or wherein between about 4.0 and about 5.0, or wherein the pH of the TEC capture buffer is pH 4, 5, 6 or 7 or fractional numbers therebetween.

Still other embodiments of the kits contain TEC-release buffers having a pH of less than about 7. In certain embodiments, the TEC-release buffers of the kits have a pH of about 7, 6, 5, 4, 3, 2 or 1, including fractional numbers therebetween. In some embodiments of the kits, each TEC release buffer has an ion (e.g., salt) concentration of is about 1000 mmole/L or less. In other embodiments of the kits, a molar concentration of ion in the first TEC release buffer is at least about 10 mmole/L less than a molar concentration of ion in the second TEC release buffer. In still other embodiments of the kits, a molar concentration of ion in the second TEC release buffer is at least about 10 mmole/L less than a molar concentration of ion in a third or additional TEC release buffer.

Other aspects of the kits use an ionizable ligand which is a heterocyclic amine. In still other embodiments, the ionizable ligand in the kit is histamine. In still other embodiments, the ionizable ligand in the kit is 4-(2-amino)pyridine, 3-(2-amino)pyridine, 2-(2-amino)pyridine, pyridine or imidazole.

Yet other embodiments of the kits contain as the solid support a bead, a magnetic bead, or a microfluidic chip. In some embodiments, the solid support further comprises free carboxylic acid functional groups. In some embodiments, the ratio of free carboxylic acids to TEC ligand bound to the solid support is between about 1 to 9 and about 1 to 3. In other embodiments, the ratio of free carboxylic acids to TEC ligand bound to the solid support is between about 1 to 3 and about 1 to 1.

In various other embodiments, the kit comprises instructions and physical and chemical components for preparing the TEC-capture buffer, and/or instructions and physical and chemical components for preparing one or more TEC-release buffers; and/or instructions for size separation of polyanions using the TEC-capture buffer and one or more TEC-release buffers.

Still other embodiment of methods described herein is a method of normalizing a concentration of polyanions from a plurality of samples. This method comprises providing a plurality of samples comprising polyanions, wherein at least one of the plurality of samples has a different concentration of polyanions than the other samples. The method also provides for each of the plurality of samples, a substantially similar amount of a solid support covalently bound to a plurality of tunable electrostatic capture (“TEC”) ligands, wherein the TEC ligands comprise an ionizable ligand which is positively charged at a pH less than a pKa of the ionizable ligand and neutral at a pH greater than the pKa of the ionizable ligand. Another step of the method is capturing an amount of polyanions in each of the plurality of samples by mixing the fixed amount the solid supports with each of the plurality of samples in a TEC capture buffer under substantially similar conditions to form a plurality of mixtures, wherein each of the plurality of mixtures has a pH less than the pKa of the ionizable ligand under conditions to allow the TEC ligands to reversibly bind to the polyanions.

Still another step of the method involves isolating the solid supports from each of the plurality of mixtures and keeping each isolated solid support separate; and releasing the amount of polyanions from the TEC ligands covalently bound to the each of the solid supports isolated in the preceding step by contacting the solid supports with a TEC elution buffer having a pH greater than the pKa of the ionizable ligand.

In various embodiments, this method is designed for use with a sample of biological components and polyanions, particularly wherein the polyanions comprise nucleic acids and/or wherein the nucleic acids are DNA or RNA.

In other embodiments of this method the amount of polyanions captured in the method is predetermined and/or the samples comprise a concentration of polyanions of at least about the predetermined amount of polyanions captured.

In another embodiment, a method of preparing a solid support for binding polyanions is provided, which comprises providing a solid support comprising at least one surface comprising carboxylic acid functional groups. This method further involves passivating (i.e., rendering non-reactive) surface ionizable groups of the solid support. A plurality of tunable electrostatic capture (“TEC”) ligands are coupled to the at least one surface wherein the TEC ligands comprise an ionizable ligand which is positively charged at a pH less than the pKa of the ionizable ligand and neutral at a pH greater than the pKa of the ionizable ligand. In certain embodiments, this method is designed for binding polyanions which are nucleic acids, including without limitation, DNA and RNA.

In other embodiments of this method, the ratio of free carboxylic acid functional groups to bound TEC ligand after the coupling step is between about 1 to 9. In another embodiment, that ratio is about 1 to 3. In still other embodiments of the method, the ratio of free carboxylic acid functional groups to bound TEC ligand after the coupling step is between about 1 to 3. In other embodiments of the method, the ratio of free carboxylic acid functional groups to bound TEC ligand after the coupling step is about 1 to 1. In still another embodiment, this method uses a solid support a plurality of resin beads.

The “tunability” of the TEC system is in Example 26 below, wherein the number and types of beads are expanded, as is the buffer compositions and optimization of TEC capture (binding) buffers that can be diluted in the presence of high (100 mM or greater) amounts of EDTA with the indicated pH adjustments. This example additional discusses capture of very large (10,000 base pairs) gDNA, among other optimizations.

Further details concerning embodiments of TEC ligands, Tec capture buffers, TEC release buffers, TEC wash and elution buffers, and methods of using same are provided in the examples below. All components of the examples below are considered as further description of the methods and compositions described above.

### EXAMPLES

The examples are provided below to facilitate a more complete understanding of the invention. The following examples illustrate the exemplary modes of making and practicing the invention. However, the scope of the invention is not limited to specific embodiments disclosed in these Examples, which are illustrative only, since alternative methods can be utilized to obtain similar results.

### Example 1: Purification of Nucleic Acids with TEC Beads

TEC magnetic beads were prepared from carboxy-modified magnetic beads obtained from Thermo Scientific. The beads were washed into N,N-dimethylformamide (DMF). The carboxylate residues on the bead surface were reacted with HBTU (2-(1H-Benzotriazole-1-yl)-1,1,3,3-tetramethyluronium hexafluorophosphate) in the presence of N,N-diisopropylethylamine to afford activated benzotriazole esters. The molar ratio of HBTU was modulated to afford sub-quantitative coupling when required.

The surface-activated beads were then reacted with histamine and N,N-diisopropylethylamine in DMF and the conjugation was allowed to proceed for about 1 hour. Following conjugation, the beads were washed with DMF followed by 50 mM sodium acetate, pH 4.0 with 0.1% Tween-20.

A sample containing nucleic acids was mixed 9:1 with 10× binding (capture) buffer (500 mM sodium acetate, pH 4.0 with 0.1% Tween-20) and 10 to 20 microliters of TEC magnetic beads. The mixture was briefly mixed via vortex or gentle pipetting. No additional incubation was needed. The magnetic beads were collected for 30 to 60 seconds and the supernatant was discarded. The beads were washed with 2×20 bead volumes (200 to 400 microliters) of TEC wash buffer (10 mM sodium acetate, pH 6.0, 0.01% Tween-20). The nucleic acids were eluted by adding 2 bead volumes (20 to 40 microliters) of either Elution Buffer (“EB”, which is 10 mM Tris-HCl, pH 8.3; Biotek) or TEC elution buffer (10 mM Tris-HCl, pH 9.0, 0.01% Tween-20). Elution was slightly more efficient with TEC elution buffer, and the pH of the final solution can be lowered to 8.3 by the addition of 1 M Tris.HCl, pH 7.5 (100×).

### Example 2: Large Scale TEC Synthesis

The synthesis was run on 4×10 ml scale. All mixing operations were performed on a platform shaker at 250 rpm with conical tubes standing vertically. All magnetic collections were performed with a 50 ml Magnetic Separation Rack (NEB). Thermo Carboxy-Modified SpeedBeads (dsMG-CM) were used as a magnetic bead base. The certificate of analysis for the beads indicated that the solution is 5.09% solids (50.9 mg/ml) and the carboxyl content is 0.4574 mmol/g. Each reaction was 5 ml.

TABLE 1
Thermo Carboxy-Modified SpeedBeads for TEC Synthesis

| | Scale | Loading | Amount | % | Volume |
|---|---|---|---|---|---|
| Reagent | (μmol) | (mmol/g) | (mg) | Solids | (μl) |
| dsMG-CM | 45.74 | 0.4574 | 100.00 | 5.09 | 1964.6 |
| (Thermo) | | | | | |

4×1965 μl (100 mg each) of the beads were transferred to clean 50 ml conical tubes. 8 ml Milli-Q water was added and the beads were magnetically collected for 5 minutes and the supernatants discarded. 10 ml DMF (EMD Millipore) was added and the mixture shaken at 250 rpm for 5 minutes. The beads were again magnetically collected for 5 minutes and the supernatant discarded. The DMF wash was repeated for a total of 2×10 ml washes. To cap residual surface ionizable groups and passivate the resin, 7.8 ml DMF (EMD Millipore), 2 ml acetic anhydride (Sigma), and 200 μl DIEA (Sigma) was added to each conical tube and the tubes were placed vertically in a rack and shaken on a platform shaker at 250 rpm for 30 minutes at room temperature. The beads were again magnetically collected for 10 minutes and the supernatants discarded. Note that after capping, the beads became sluggish to collect (especially during the DMF washes) and more time (e.g., 10 min) can be allowed for magnetic collection. The supernatant was brown from the capping mixture. 10 ml DMF (EMD Millipore) was added and the mixture was shaken at 250 rpm for 5 minutes, the beads were magnetically collected for 10 minutes and the supernatants discarded. The DMF washes were repeated 3 more times for a total of 4×10 ml washes.

Resin activation and coupling. The coupling reagents were prepared as follows.

TABLE 2
Preparation of HBTU solution

| | | D | Mass | Volume | |
|---|---|---|---|---|---|
| Reagent | MW | (g/ml) | (mg) | (μl) | Conc (mg/μl) |
| HBTU (Nova | 379.25 | 1 | 91.5 | 1000 | 0.0915 |
| Biochem) | | | | | |

91.5 mg of HBTU was weighed and dissolved in 910 μl DMF (1000 μl final volume), yielding a concentration of 0.0915 mg/μl.

TABLE 3
Preparation of HBTU and DIEA

| | | D | | | | Amt | |
|---|---|---|---|---|---|---|---|
| | | (g/ | Scale | | | (mg | Actual |
| Reagent | MW | ml) | (μmol) | EQ | Couplings | or μl) | (mg/μl) |
| HBTU | 379.25 | 1 | 45.74 | 1.1 | 1 | 19.082 | 208.54 |
| (Nova | | | | | | | |
| Biochem) | | | | | | | |
| DIEA | 129.24 | 0.742 | 45.74 | 3 | 1 | 23.90 | 23.90 |
| (Sigma) | | | | | | | |

The indicated amount of HBTU and DIEA was diluted into 4.8 ml DMF (adding DIEA last) and mixed immediately upon base addition. The final volume was ˜5 ml. The solutions were mixed well and rotated at room temperature for 5 minutes to activate the carboxylic acids. The histamine was prepared beforehand by dissolving in 19.75 ml DMF (EMD Millipore) for a 20 ml final volume.

TABLE 4
Preparation of histamine

| | | D | Mass | Volume | Conc |
|---|---|---|---|---|---|
| Reagent | MW | (g/ml) | (mg) | (μl) | (mg/μl) |
| Histamine (Sigma) | 111.15 | 1 | 252.6 | 20000 | 0.0126 |

TABLE 5
Preparation of histamine and DIEA

| | | | | | | Amt | |
|---|---|---|---|---|---|---|---|
| | | D | Scale | | | (mg | Actual |
| Reagent | MW | (g/ml) | (μmol) | EQ | Couplings | or μl) | (mg/μl) |
| Histamine | 111.15 | 1 | 45.74 | 10 | 1 | 50.84 | 4025.34 |
| (Sigma) | | | | | | | |
| DIEA | 129.24 | 0.742 | 45.74 | 10 | 1 | 79.67 | 79.67 |
| (Sigma) | | | | | | | |

After a 5-minute activation, the histamine (Sigma) and DIEA (Sigma) was added to the beads, and the final volume was adjusted to 10 ml with DMF so that the final bead concentration was 1%. This mixture was shaken at 250 rpm at room temperature for 1 hour. The beads were magnetically collected for 5 minutes and the supernatants discarded. Note that after conjugation, the beads collected more readily. 10 ml DMF (EMD Millipore) was added and the mixture was shaken at 250 rpm for 5 minutes. The beads were magnetically collected for 5 minutes and the supernatants discarded. The DMF washes were repeated (with shaking) 3 more times for a total of 4×10 ml washes, and 10 ml TE buffer pH 8.0 (Amresco) was added. The tubes were vortexed well to mix and quickly spun at 1,000 g. The beads were magnetically collected for 5 minutes and the supernatant discarded. The TE pH 8.0 wash was repeated three more times for a total of 4×10 ml washes. The tubes were quickly spun at 1,000 g and the resins magnetically collected for an additional 5 minutes and any remaining liquid was discarded. 9.5 ml TE pH 8.0 was added to each tube for a final volume of 10 ml each, and these were combined and stored at 4° C.

### Example 3: TEC Sub-Quantitative Coupling

The magnetic resin was prepared using carboxy-modified SpeedBeads. The following coupling procedure is for 0.5 ml of 1% carboxylate beads and used the Thermo Carboxy-Modified SpeedBeads (dsMG-CM) as a magnetic bead base. The certificate of analysis for the beads indicated that the solution was 4.99% solids (49.9 mg/ml) and the carboxyl content was 0.4404 mmol/g.

TABLE 6
Thermo Carboxy-Modified SpeedBeads for
TEC Sub-Quantitative Coupling

| | Scale | Loading | Amount | % | Volume |
|---|---|---|---|---|---|
| Reagent | (μmol) | (mmol/g) | (mg) | Solids | (μl) |
| dsMG-CM | 2.198 | 0.4404 | 4.99 | 4.99 | 100.0 |
| (Thermo) | | | | | |

8×100 μl (4.99 mg each) of the beads was transferred to a clean microfuge tube, and the beads were magnetically collected and the supernatant discarded. The beads were washed with 0.5 ml nuclease-free water (PW1 Illumina), and the last wash was discarded and the beads given a quick spin. The beads were returned to the magnet and any residual water that collected at the bottom of the tube was removed. 0.5 ml DMF (EMD Millipore) was added and vortexed well, given a quick spin to magnetically collect the beads. The DMF wash was repeated for a total of 2×0.5 ml washes. To cap residual surface ionizable groups and passivate the resin, 780 μl DMF (EMD Millipore), 200 μl acetic anhydride (Sigma), and 20 μl DIEA (Sigma) was added to each conical. The tubes were rotated for 30 minutes at room temperature, given a quick spin, the beads and magnetically collected, and the supernatants discarded. Note that after capping the beads became sluggish to collect (especially during the DMF washes) and more time (e.g., 10 min) can be allowed for magnetic collection. The supernatant is brown from the capping mixture. 0.5 ml DMF (EMD Millipore) was added, the mixtures vortexed well, given a quick spin and the beads magnetically collected. The DMF washes were repeated 3 more times for a total of 4×0.5 ml washes.

The amounts of coupling reagents for amine coupling for TEC0, TEC20, TEC25, TEC30, TEC35, TEC40, TEC45, and TEC50 beads used are shown in Table 7.

TABLE 7
Coupling reagents for TEC Sub-Quantitative Coupling

| | | D | | | | Amt | |
|---|---|---|---|---|---|---|---|
| | | (g/ | Scale | | | (mg | Actual |
| Reagent | MW | ml) | (μmol) | EQ | Couplings | or μl) | (mg/μl) |
| HBTU | 379.25 | 1 | 2.198 | 0 | 1 | 0.000 | 0.00 |
| (Nova | | | | | | | |
| Biochem) | | | | | | | |
| HBTU | 379.25 | 1 | 2.198 | 0.20 | 1 | 0.167 | 6.04 |
| (Nova | | | | | | | |
| Biochem) | | | | | | | |
| HBTU | 379.25 | 1 | 2.198 | 0.25 | 1 | 0.208 | 7.55 |
| (Nova | | | | | | | |
| Biochem) | | | | | | | |
| HBTU | 379.25 | 1 | 2.198 | 0.30 | 1 | 0.250 | 9.06 |
| (Nova | | | | | | | |
| Biochem) | | | | | | | |
| HBTU | 379.25 | 1 | 2.198 | 0.35 | 1 | 0.292 | 10.57 |
| (Nova | | | | | | | |
| Biochem) | | | | | | | |
| HBTU | 379.25 | 1 | 2.198 | 0.40 | 1 | 0.33 | 12.08 |
| (Nova | | | | | | | |
| Biochem) | | | | | | | |
| HBTU | 379.25 | 1 | 2.198 | 0.45 | 1 | 0.38 | 13.59 |
| (Nova | | | | | | | |
| Biochem) | | | | | | | |
| HBTU | 379.25 | 1 | 2.198 | 0.50 | 1 | 0.42 | 15.10 |
| (Nova | | | | | | | |
| Biochem) | | | | | | | |
| DIEA | 129.24 | 0.742 | 2.198 | 3 | 1 | 1.15 | 1.15 |
| (Sigma) | | | | | | | |

13.8 mg of HBTU was weighed out and dissolved in 490 μl DMF (500 μl final volume), yielding a concentration of 0.0276 mg/μl. The indicated amount of HBTU and DIEA was diluted into 330 μl DMF, adding DIEA last. The tubes were mixed well and rotated at room temperature for 5 minutes to activate the carboxylic acids.

The amounts of amine compounds used for amine coupling are shown in Table 8.

TABLE 8
Amine compounds for TEC Sub-Quantitative Coupling

| | | | | | | Amt | Actual |
|---|---|---|---|---|---|---|---|
| | | D | Scale | | | (mg | (mg |
| Reagent | MW | (g/ml) | (μmol) | EQ | Couplings | or μl) | or μl) |
| Histamine | 111.15 | 1 | 2.198 | 10 | 8.1 | 19.79 | 19.80 |
| (Sigma) | | | | | | | |
| DIEA | 129.24 | 0.742 | 2.198 | 10 | 8.1 | 31.01 | 31.00 |
| (Sigma) | | | | | | | |

The histamine and DIEA were dissolved in 600 μl DMF and the final volume was adjusted to 1215 μl with DMF. After the 5-minute bead activation, 150 μl of the histamine solution was added to the beads. The final volume of solution added was approximately 500 μl (beads at 1%) and the reactions were rotated at room temperature for 1 hour. The beads were magnetically collected and the supernatant discarded, and the beads were washed with 2×1 ml DMF (Sigma), followed by 3×1 ml TE pH 8 (Amresco). After the last wash, the beads were spun quickly, returned to the magnet and any residual TE was removed. An additional 480 μl TE was added to each bead sample to a final volume of 500 μl and stored at 4° C.

### Example 4: DNA Damage Assessment

To demonstrate that the low pH buffer used in TEC binding does not damage target DNA via acid-catalyzed depurination, the following experiment was performed with the following buffer sets: (1) 10×pH 4.8 binding buffer: 1 M NaOAc (Ambion), 1 M NaCl (Ambion), 0.1% Tween-20 (Enzo Life Sciences), pH 4.8; (2) 10×pH 4.0 binding buffer: 1 M NaOAc (Ambion), 1 M NaCl (Ambion), 0.1% Tween-20 (Enzo Life Sciences), pH 4.0; (3) TEC wash buffer: 10 mM sodium phosphate (Amresco), 0.01% Tween-20 (Enzo Life Sciences), pH 6.0; (4) TEC Elute: EB (Biotek) was supplemented with 0.01% Tween-20 (Enzo Life Sciences).

4.14 μl (250 ng) of an Illumina sequencing library was aliquoted into 24 microfuge tubes. Master mixes of pH 4.0 and 4.8 binding buffer (15 reactions, each with 2.5 μl binding buffer and 15.86 μl Milli-Q water) were made. 18.36 μl of the binding buffers were added to the DNA samples, adding pH 4.0 to 12 samples and pH 4.8 to the other 12, and mixed well.

After 5, 15, 30, and 60 minutes, 3 samples from each buffer set were processed in the following way. 2.5 μl TEC beads were added, vortexed, spun quickly, and magnetically collected. The liquid was discarded and 100 μl TEC Wash was added and the mixture was vortexed, spun quickly, and the beads magnetically collected. The wash was repeated for a total of 2 washes. The beads were resuspended in 20 μl TEC Elute and mixed well. The beads were magnetically collected and the eluate transferred to a clean PCR plate.

In addition to the acid-treated samples, 3×4.14 μl aliquots (brought to 20 μl with TEC Elute) were made for non-treated controls.

For DNA Quantitation, the plate was mixed well (2000 rpm for 30 sec) and spun quickly to collect all liquid. 81×198 μl Qubit dsDNA HS aliquots were made in a 800 μl storage plate using a P200 multichannel pipette. 3×2 μl of each eluate was added and quantified via Qubit. Summary data (average of 3 reads) is shown in Table 9.

TABLE 9
DNA Quantitation

| Sample | Av Conc (ng/μl) | SD |
|---|---|---|
| Input A | 13.30 | 0.20 |
| Input B | 12.70 | 0.35 |
| Input C | 12.70 | 0.62 |
| pH 4.0 5′ A | 10.67 | 0.31 |
| pH 4.0 5′ B | 11.17 | 0.55 |
| pH 4.0 5′ C | 10.83 | 0.15 |
| pH 4.8 5′ A | 10.73 | 0.15 |
| pH 4.8 5′ B | 9.27 | 0.12 |
| pH 4.8 5′ C | 10.43 | 0.31 |
| pH 4.0 15′ A | 10.63 | 0.06 |
| pH 4.0 15′ B | 10.97 | 0.06 |
| pH 4.0 15′ C | 12.33 | 1.69 |
| pH 4.8 15′ A | 12.57 | 1.42 |
| pH 4.8 15′ B | 11.03 | 0.23 |
| pH 4.8 15′ C | 10.07 | 0.28 |
| pH 4.0 30′ A | 10.09 | 0.11 |
| pH 4.0 30′ B | 10.17 | 0.21 |
| pH 4.0 30′ C | 10.57 | 0.25 |
| pH 4.8 30′ A | 10.87 | 0.75 |
| pH 4.8 30′ B | 11.93 | 1.22 |
| pH 4.8 30′ C | 12.00 | 0.70 |
| pH 4.0 60′ A | 11.43 | 0.55 |
| pH 4.0 60′ B | 10.11 | 0.88 |
| pH 4.0 60′ C | 10.57 | 0.15 |
| pH 4.8 60′ A | 10.80 | 0.35 |
| pH 4.8 60′ B | 9.96 | 0.21 |
| pH 4.8 60′ C | 9.30 | 0.15 |

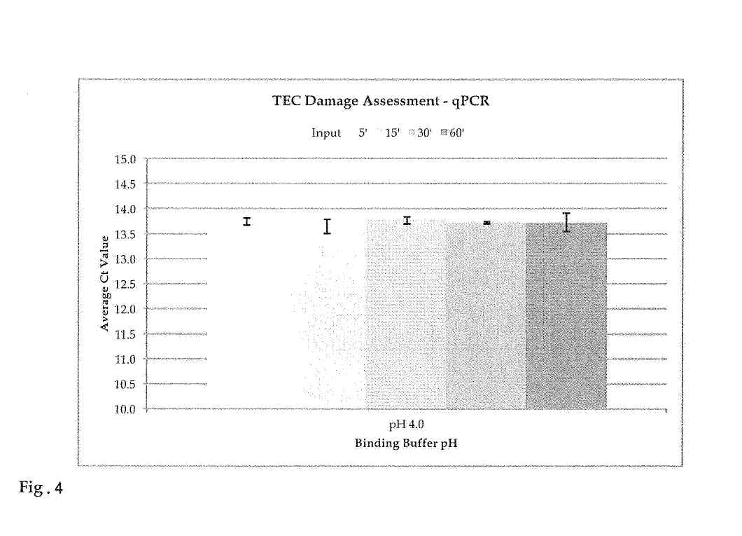

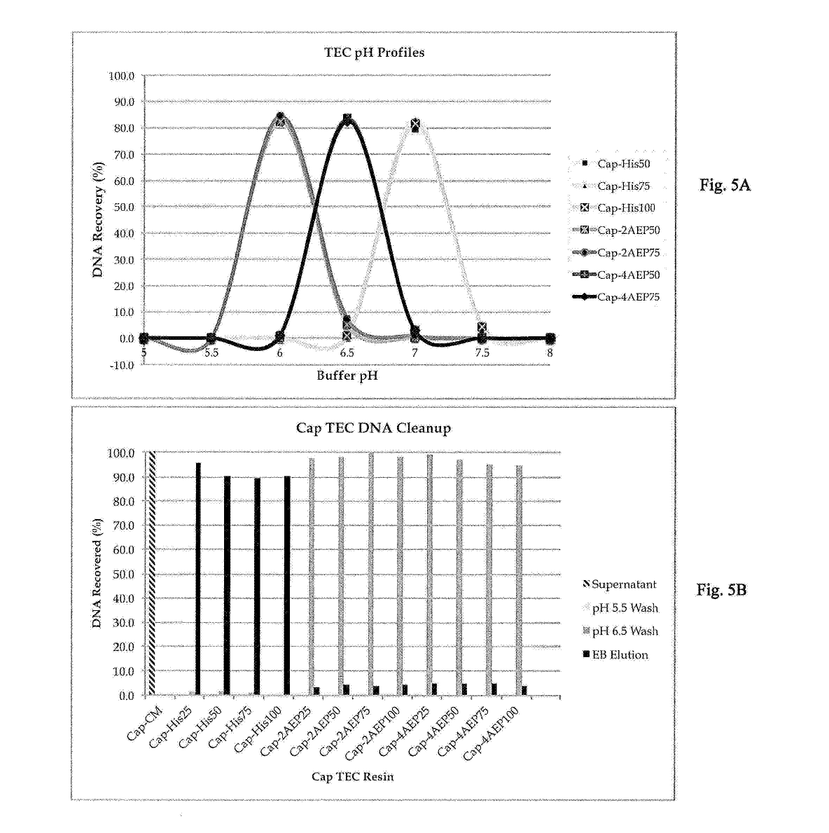

For plate normalization, using the average concentration values in Table 9 above, 1 μl of each sample was diluted with Milli-Q water to 1 ng/μl in a fresh PCR plate. The plates were mixed well, spun down, and qPCR (Kapa Biosystems, Library Quantification Kit) was performed and the results shown in FIG. 4. DNA damage results in less efficient PCR and subsequently higher Ct values. As shown in FIG. 4, the pH 4.0 data was nearly flat, with tight error bars and a nearly uniform Ct value compared to input, indicating no detectable damage to the DNA library. Thus, under these conditions, no change in Ct values were observed between input and test samples, indicating that the TEC binding system is safe for DNA, even with incubation periods of up to 1 hour.

### Example 5: His and AEP Modified TEC Bead pH Elution Profile

Histamine- and 2-, 3-, and 4-(2-Aminoethyl)pyridine (AEP) modified TEC beads with and without surface passivation were employed for DNA capture followed by resin washing at various incremental pH values, using the following buffers: 10×pH 4.0 binding buffer: 1 M NaOAc (Ambion), 1 M NaCl (Ambion), 0.1% Tween-20 (Enzo Life Sciences), pH 4.0. TEC wash buffers: 10 mM sodium phosphate (Amresco), 0.01% Tween-20 (Enzo Life Sciences), pH 4.5, 5.0, 5.5, 6.0, 6.5, 7.0, 7.5, or 8.0. EB (Biotek) was supplemented with 0.01% Tween-20 (Enzo Life Sciences).

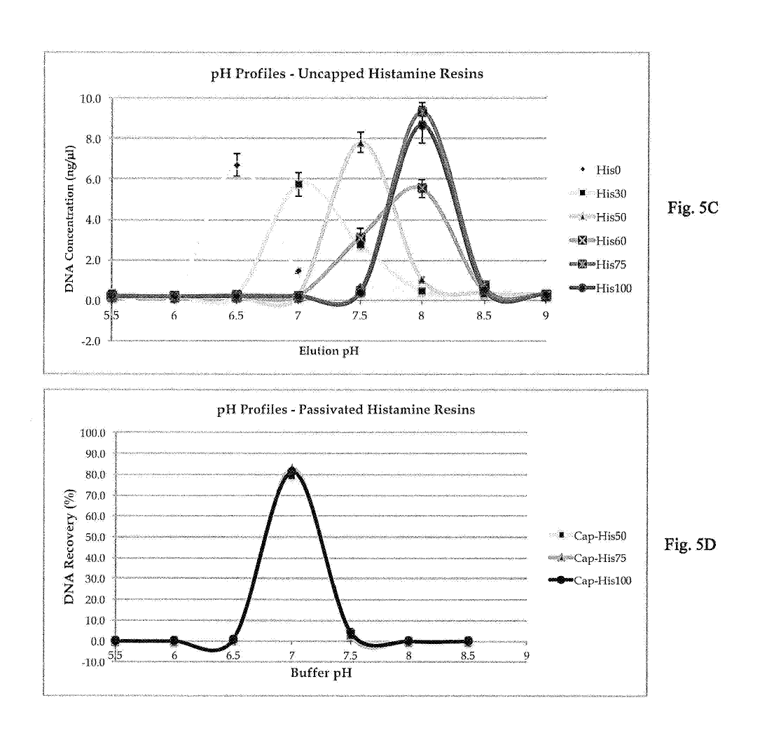

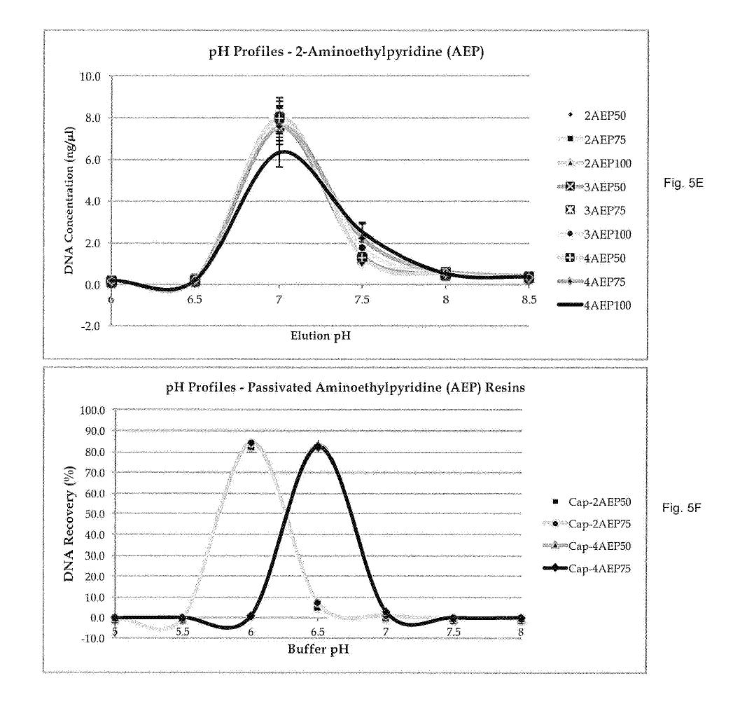

5 μl Cap-His50, Cap-His75, Cap-His100, Cap-2AEP75, Cap-2AEP100, Cap-4AEP75, or Cap-4AEP100 were aliquoted into a 200 μl Bio-Rad PCR plate. A pool of sheared DNA was created by shearing Coriell sample NA12878 DNA at 150, 350, and 550 bp and equally mixing all sheared samples. The final nucleic acid concentration of the pool was 30.6 ng/μl. The pool was aliquoted and stored at −20° C. until needed. 5 μl 10×TEC binding buffer, 8.2 μl fragmentation pool (250 ng, at 30.6 ng/μl), and 31.8 μl Milli-Q water was added and mixed via pipette. The beads were magnetically collected and the supernatant discarded. The beads were washed with 100 μl TEC wash buffer pH 4.5 and the supernatants discarded, and then sequentially washed with 10 μl of each wash buffer from pH 5.0-8.0, increasing in pH with each wash, and the eluates saved for analysis. Each eluate was quantified with Qubit dsDNA HS (Life Technologies) using 2 μl of eluate, and the results shown in FIGS. 5A-5F.

The pH elution profile for each resin at different ligand concentrations is shown in FIG. 5A, with ligand-types clearly clustering according to type. FIG. 5B shows the results of initial cleanup reactions, where DNA was bound to various TEC resins, washed with two different pH buffers (5.5 and 6.5), and eluted with buffer at pH 8.3, and DNA capture and recovery at each condition is shown. As can be seen in FIG. 5B, histamine ligands with higher pKa values retain DNA through the pH 5.5 and 6.5 washes, whereas the AEP resins with lower pKas show elution at pH 6.5. FIG. 5C shows that non-passivated histamine (TEC) resins demonstrated an increase in pH-dependent elution with increasing concentration of surface-conjugated ligand. FIG. 5D shows that, after surface passivation with acetic anhydride, histamine resins demonstrated a ligand density-independent pH elution profile. FIG. 5E shows that non-passivated AEP resins show a slightly broad pH-dependent elution which is independent of the concentration or isomer of surface-conjugated AEP ligand. FIG. 5F shows that, after surface passivation with acetic anhydride, AEP resins show a narrower, ligand density-independent pH elution profile, and differences between isomers (e.g. ortho vs para substitution) is apparent.

### Example 6: CAP-TEC Cleanup

Initial cleanup reactions of DNA bound to various TEC resins washed with two different pH buffers (5.5 and 6.5), and eluted with buffer at pH 8.3 were performed with the following buffers: 10×pH 4.0 binding buffer: 1 M NaOAc (Ambion), 1 M NaCl (Ambion), 0.1% Tween-20 (Enzo Life Sciences), pH 4.0. TEC wash buffers: 10 mM sodium phosphate (Amresco), 0.01% Tween-20 (Enzo Life Sciences), pH 5.5 or 6.5. EB (Biotek) was supplemented with 0.01% Tween-20 (Enzo Life Sciences).

5 μl Cap-His25, Cap-His50, Cap-His75, Cap-His100, Cap-2AEP25, Cap-2AEP50, Cap-2AEP75, Cap-2AEP100, Cap-4AEP25, Cap-4AEP50, Cap-4AEP75, or Cap-4AEP100 were aliquoted into a 200 μl Bio-Rad PCR plate. A pool of sheared DNA was created by shearing Coriell sample NA12878 DNA at 150, 350, and 550 bp and equally mixing all sheared samples. The final nucleic acid concentration of the pool was 30.6 ng/μl. The pool was aliquoted and stored at −20° C. until needed. 5 μl 10×TEC binding buffer, 8.2 μl fragmentation pool (250 ng, at 30.6 ng/μl), and 31.8 μl Milli-Q water was added and mixed via pipette. The beads were magnetically collected and the supernatant discarded. The beads were washed with 100 μl TEC wash buffer pH 5.0 and the supernatants discarded, and the beads were then sequentially washed with 10 μl of each wash buffer (pH 5.5 then pH 6.5) followed by 10 μl of EB, and the eluates were saved for analysis. Each eluate was quantified with Qubit dsDNA HS (Life Technologies) using 2 μl of eluate, and the results are shown in FIG. 5E. As indicated in FIG. 5E, histamine ligands (which have higher pKa values) retained DNA through the pH 5.5 and 6.5 washes, whereas the AEP resins (which have lower pKa values) showed elution at pH 6.5.

### Example 7: TEC Recovery Efficiency

The efficiency of various TEC ligands to capture DNA was demonstrated by binding purified DNA to various TEC ligand-modified resins at low pH, washing them with an intermediate pH specific for each TEC ligand, and eluting at pH 8.3 using the following buffers: 10×pH 4.8 binding buffer: 0.5 M NaOAc (Ambion), 1% Tween-20 (Enzo Life Sciences), pH 4.8. Wash buffers: sodium phosphate (Amresco), Tween-20 (Enzo Life Sciences), and NaCl (Ambion): 10 mM sodium phosphate, 0.01% Tween-20, pH 5.3, pH 5.8 and pH 6.3. EB (Biotek) was supplemented with 0.01% Tween-20 (Enzo Life Sciences).

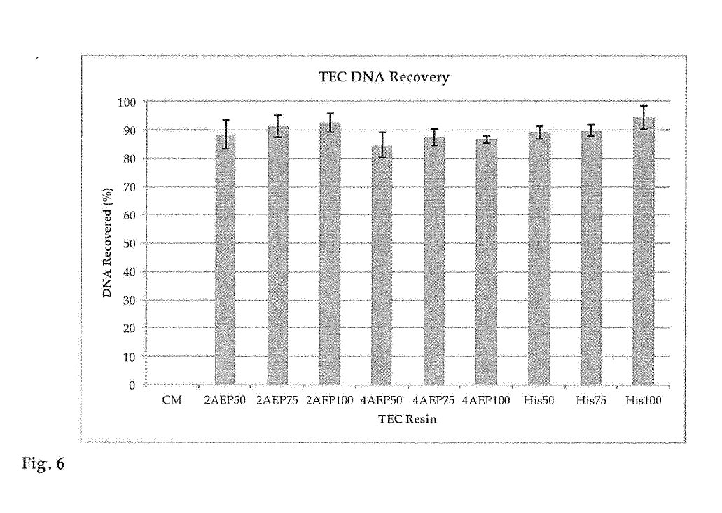

3×5 μl of Cap-CM, Cap-His50, Cap-His75, Cap-His100, Cap-2AEP50, Cap-2AEP75, Cap-2AEP100, Cap-4AEP50, Cap-4AEP75, and Cap-4AEP100 were aliquoted into a 200 μl BioRad PCR plate. 50 μl final volume binding reactions were created using 10×pH 4.8 binding buffer, Milli-Q water, and 2 μl of a purified, PCR-amplified sequencing library (58 ng/μl). The beads were collected and the supernatant discarded. The beads were then washed by mixing via pipette with 2×100 μl wash buffer at the following pH values: Cap-2AEP, pH 5.3; Cap-4AEP, pH 5.8; Cap-His, pH 6.3, and all samples were eluted with 2×10 μl EB/Tween-20. The eluates were combined, and 2 μl was used for quantitation via Qubit dsDNA HS and the results shown in FIG. 6. As indicated in FIG. 6, all resins showed efficient capture, and each ligand type showed a slight enhancement with increasing amounts of ligand conjugated to the bead surface.

### Example 8: TEC SPRI PCR Cleanup

Methodologies using TEC beads and SPRI beads for post-PCR DNA recovery were compared using the following buffers: 10×pH 4.0 binding buffer: 1 M NaOAc (Ambion), 1 M NaCl (Ambion), 0.1% Tween-20 (Enzo Life Sciences), pH 4.0. EB (Biotek) was supplemented with 0.01% Tween-20 (Enzo Life Sciences). Wash buffer used sodium phosphate (Amresco), Tween-20 (Enzo Life Sciences): 10 mM sodium phosphate, 0.01% Tween-20, pH 6.0.

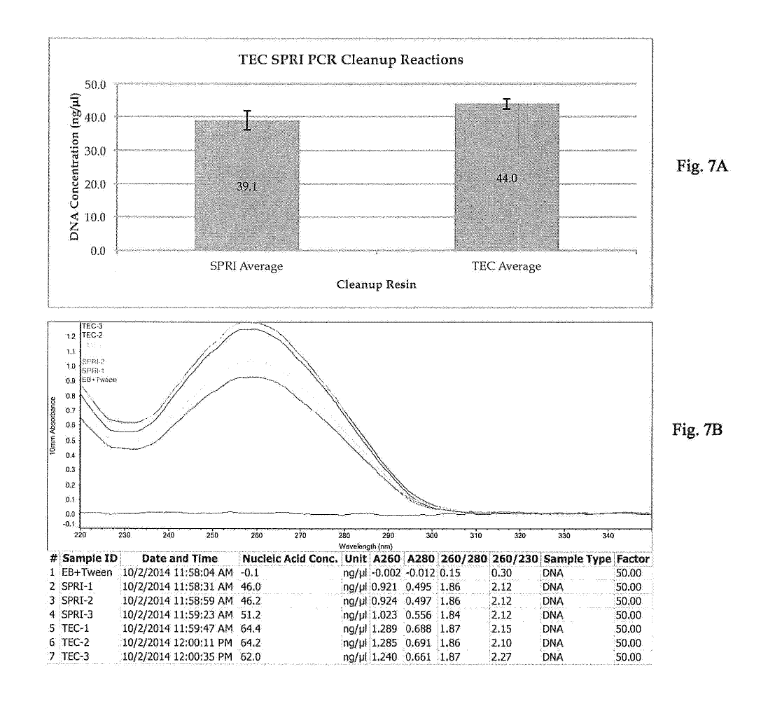

PCR-amplified DNA was divided into 6 equal portions and purifications were performed in triplicate with either SPRI or TEC resin. For the TEC resin, 3×6 μl of Cap-His100 was aliquoted into a 200 μl Bio-Rad PCR plate. For each reaction, 15 μl of a PCR-amplified sequencing library, 4.5 μl 10× binding buffer, and 19.5 μl Milli-Q water was combined. The reactions were mixed via pipette, spun quickly, and the beads collected. The supernatants were discarded. The beads were washed with 2×45 μl wash buffer by mixing via pipette followed by a quick spin and eluted with 15 μl EB/Tween-20. For the SPRI resin, Sample Purification Beads (Illumina) was used. 15 μl of the PCR product was diluted with 30 μl of beads (2:1 ratio) and the Beckman Coulter protocol for Ampure XP PCR cleanup (washing with 150 μl 75% ethanol) was followed, and the products eluted in 15 μl EB (Biotek) supplemented with 0.01% Tween-20 (Enzo Life Sciences). 1 μl of each TEC resin or SPRI resin eluates was used for quantitation via Qubit dsDNA HS, and all samples were run on the NanoDrop to assess DNA purity. The results are shown in FIGS. 7A and 7B. As indicated in FIG. 7A, TEC resins consistently recovered more material as compared to the SPRI resin. FIG. 7B shows the NanoDrop traces for the replicates of FIG. 7A. As shown in FIG. 7B, the ratio of absorbance at 260 nm to 230 nm for all samples were over 2, indicating that pure material was recovered.

### Example 9: TEC SPRI RNA Cleanup

Methodologies using TEC beads and SPRI beads for recovery of RNA from a sample were compared using the following buffers: 10×TEC Binding Buffer: 1 M NaOAc (Ambion), 1 M NaCl (Ambion), 0.1% Tween-20 (Enzo Life Sciences), pH 4.0. TEC Wash Buffer pH 6.0: 10 mM sodium phosphate (Amresco), 0.01% Tween-20 (Enzo Life Sciences), pH 6.0. EB (Biotek) is supplemented with 0.01% Tween-20 (Enzo Life Sciences).

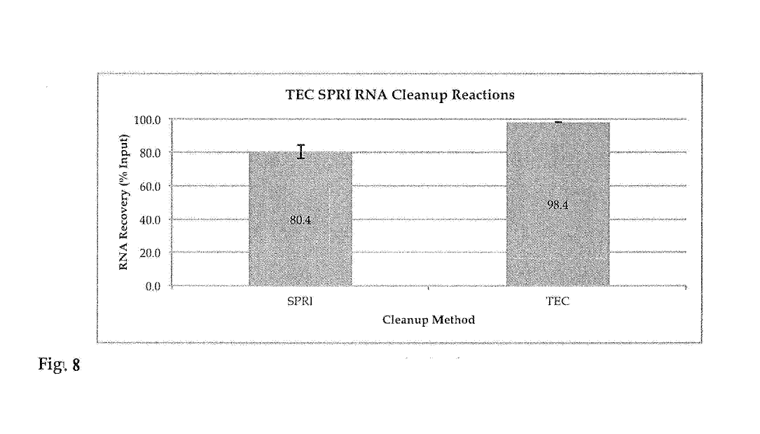

For the TEC resin, 2×5 μl Cap-His100 was aliquoted into 1.5 ml microfuge tubes. For each reaction, 5 μl of a standard RNA sample (Universal Human Reference or “UHR”) (estimated at 150 ng/μl), 2.5 μl 10× binding buffer and 17.5 μl Milli-Q water was combined and mixed by vortexing, spun quickly, the beads collected and the supernatants discarded. The beads were then washed by mixing via vortex followed by a quick spin with 2×100 μl wash buffer. The RNA was eluted from the beads with 10 μl EB/Tween-20 at room temperature. For the SPRI beads, Sample Purification Beads (Illumina) were used. 5 μl of UHR RNA were diluted into 5 μl Milli-Q water and 20 μl of beads (2:1 ratio), and the Beckman Coulter protocol for Ampure XP PCR cleanup (washing with 150 μl 75% ethanol) was followed. The RNA was eluted from the beads in 10 WEB (Biotek) supplemented with 0.01% Tween-20 (Enzo Life Sciences). All TEC-resin and SPRI-resin samples were analyzed on the NanoDrop (1.5 μl each, n=3), and portions of all samples were frozen at −80° C. in case a BioAnalyzer trace was needed. As shown in FIG. 8, TEC resins consistently recovered more RNA than SPRI resins.

### Example 10: TEC pH Elution Profile

The pH elution profile from Cap-His100 beads was determined using the following buffers: 10×pH 4.8 binding buffer: 1 M NaOAc (Ambion), 1 M NaCl (Ambion), 0.1% Tween-20 (Enzo Life Sciences), pH 4.8. TEC wash buffer pH 6: 10 mM sodium phosphate (Amresco), 0.01% Tween-20 (Enzo Life Sciences), pH 6.0. TEC wash buffer pH 7.8: 10 mM sodium phosphate (Amresco), 0.01% Tween-20 (Enzo Life Sciences), pH 7.8. EB (Biotek) was supplemented with 0.01% Tween-20 (Enzo Life Sciences).

DNA was adsorbed to the surface of the Cap-His100 beads at low pH and washed to remove the binding buffer. The beads were sequentially washed with 10 mM sodium phosphate at increasing pH values. The low and high pH buffers were proportionally mixed to make 18 intermediate pH buffers. The overall series was 10 mM sodium phosphate (Amresco) with 0.01% Tween-20 (Enzo Life a Sciences), at pH 6.0, 6.22, 6.33, 6.45, 6.55, 6.63, 6.73, 6.80, 6.87, 6.95, 7.01, 7.07, 7.15, 7.22, 7.29, 7.37, 7.46, 7.55, 7.62 and 7.73.

Binding and elution of DNA with the Cap-His100 beads was performed as follows: 5 μl Cap-His100 was aliquoted into a 200 μl Bio-Rad PCR plate. A pool of sheared DNA was created by shearing Coriell sample NA12878 DNA at 150, 350, and 550 bp and equally mixing all sheared samples. The final nucleic acid concentration of the pool was 30.6 ng/μl. The pool was aliquoted and stored at −20° C. until needed. 5 μl 10×TEC binding buffer, 8.2 μl fragmentation pool (250 ng, at 30.6 ng/μl), and 31.8 μl Milli-Q water was added and mixed via pipette. The beads were magnetically collected and the supernatant discarded. The beads were washed with 2×150 μl TEC wash buffer pH 6 and the supernatants discarded. The resins were then sequentially washed with 10 μl of each pH buffer, increasing in pH with each wash, and the eluates saved for analysis. 2 μl of each eluate was quantified with Qubit dsDNA HS (Life Technologies). As shown in FIG. 9, DNA elution from Cap-His100 beads began around pH 6.4 and was essentially complete by pH 7.

### Example 11: TEC Size Selection Analysis

To investigate the usefulness of size selective pH elution of nucleic acids bound to TEC ligands, samples of DNA molecules from the pH elution profile analysis performed in Example 10 were subjected to electrophoretic analysis on the Agilent Bioanalyzer (High Sensitivity) chip. The DNA sizes eluted at each pH point were determined, and the results are shown in FIG. 10A. A DNA fragment pool (35-1500 bp) obtained from Example 10 was bound to TEC beads (Cap-His100) and subjected to incremental pH washes (pH 6.55-6.95). As shown in FIG. 10A, the elution profile was very broad and thus was not useful for precise size selection

### Example 12: TEC Size Selection Analysis, pH 6.48-6.60

The usefulness of size selective pH elution of nucleic acids bound to TEC ligands over a narrow pH range (6.48-6.60) was further investigated using the following buffers: 10×pH 4.8 binding buffer: 1 M NaOAc (Ambion), 1 M NaCl (Ambion), 0.1% Tween-20 (Enzo Life Sciences), pH 4.8. TEC wash buffer pH 6.48: 10 mM sodium phosphate (Amresco), 0.01% Tween-20 (Enzo Life Sciences), pH 6.0. TEC wash buffer pH 6.60: 10 mM sodium phosphate (Amresco), 0.01% Tween-20 (Enzo Life Sciences), pH 7.8. EB (Biotek) was supplemented with 0.01% Tween-20 (Enzo Life Sciences).

As in Example 10, DNA was adsorbed to the surface of Cap-His100 beads at low pH and washed to remove the binding buffer. The beads were sequentially washed with 10 mM sodium phosphate at increasing pH values. The low and high pH buffers were proportionally mixed to make intermediate pH buffers. The overall series was 10 mM sodium phosphate (Amresco) with 0.01% Tween-20 (Enzo Life a Sciences), at pH 6.48, 6.49, 6.50, 6.51, 6.52, 6.54, 6.56, 6.58, 6.59 and 6.60.

Binding and elution of DNA with the Cap-His100 beads was performed as follows: 5 μl Cap-His100 was aliquoted into a 200 μl Bio-Rad PCR plate. A pool of sheared DNA was created by shearing Coriell sample NA12878 DNA at 150, 350, and 550 bp and equally mixing all sheared samples. The final nucleic acid concentration of the pool was 30.6 ng/μl. The pool was aliquoted and stored at −20° C. until needed. 5 μl 10×TEC binding buffer, 8.2 μl fragmentation pool (250 ng, at 30.6 ng/μl), and 31.8 μl Milli-Q water was added and mixed via pipette. The beads were magnetically collected and the supernatant discarded, and the beads were washed with 2×150 μl TEC wash buffer pH 6 and the supernatants discarded. The resins were sequentially washed with 10 μl of each pH buffer, increasing in pH with each wash, and the eluates saved for analysis. The size distribution of DNA eluted at each pH step was analyzed via on-chip electrophoresis on the Agilent Bioanalyzer High Sensitivity chip. The results are shown in FIG. 10B. As indicated in FIG. 10B, even with a very fine gradation of pH, the elution profile was very broad and not particularly size selective.

### Example 13: TEC NaCl Size Selection

Nucleic acid elution from TEC resins by varying ion concentration was demonstrated with the following buffers: 10×pH 4.8 binding buffer: 1 M NaOAc (Ambion), 1 M NaCl (Ambion), 0.1% Tween-20 (Enzo Life Sciences), pH 4.8. TEC NaCl wash buffers: 10 mM sodium phosphate (Amresco), at 0, 130, 140, 150, 160, 170, 180, 190, 200, 210, 220, 230, 240, 250, 260, 270, 280, 290, 300, 310, or 320 mM NaCl (Ambion) 0.01% Tween-20 (Enzo Life Sciences), pH 6.0. TEC wash buffer: 10 mM sodium phosphate (Amresco), 0.01% Tween-20 (Enzo Life Sciences), pH 6.0. EB (Biotek) was supplemented with 0.01% Tween-20 (Enzo Life Sciences).

A defined low-molecular weight DNA ladder was bound to TEC resin (Cap-His100), and the DNA molecules were size-selectively eluted with increasing concentrations of ions (NaCl) as follows: 20×6 μl Cap-His100 was aliquoted into a 200 μl Bio-Rad PCR plate. Enough master mix was made for 21 reactions, each containing 4.5 μl 10×TEC binding buffer, 0.4 μl (200 ng) low range MW ladder (NEB), and 34.1 μl Milli-Q water. 39 μl reaction of the master mix was added to each well containing beads and mixed via pipette. The beads were magnetically collected and the supernatants discarded. The beads were washed with 2×45 μl TEC NaCl wash buffers and the supernatants discarded, with each well receiving a different salt concentration. The beads were washed with 45 μl TEC wash buffer and the supernatant discarded. 40 μl EB/Tween was added and mixed well to elute the DNA. The eluted DNA was analyzed on 2 BioAnalyzer (High Sensitivity) chips.

To analyze the data, global region tables were created: 45-70 (50 bp), 70-90 (75 bp), 90-120 (100 bp), 145-175 (150 bp), 185-240 (200 bp), 240-280 (250 bp), 290-335 (300 bp), 340 390 (350 bp), 465-555 (500 bp), 680-1070 (776 bp). The BioAnalyzer corrected area for these regions were exported, and all corrected areas were normalized to the area recovered in the 130 mM NaCl sample and all traces were graphed, and the results shown in FIG. 11A. As indicated in FIG. 11A, the ion in the wash buffers electrostatically interfered with the resin:DNA interactions. As the bound DNA molecules increased in size, more ions were required for electrostatic interference, which allowed for effective size selection.

### Example 14: TEC NaCl Binding

The binding of nucleic acids to TEC resins by varying ion concentration was demonstrated with the following buffers: 10×pH 4.8 binding buffer: 1 M NaOAc (Ambion), 1 M NaCl (Ambion), 0.1% Tween-20 (Enzo Life Sciences), pH 4.8. TEC wash buffer: 10 mM sodium phosphate (Amresco), 0.01% Tween-20 (Enzo Life Sciences), pH 6.0. EB (Biotek) was supplemented with 0.01% Tween-20 (Enzo Life Sciences).

A defined low-molecular weight DNA ladder was contacted with TEC resin (Cap-His100) as follows: 11×5 μl Cap-His100 was aliquoted into a 200 μl Bio-Rad PCR plate. A volume of 2 M NaCl (Ambion) was added to each well to bring the final NaCl concentrations to 400, 450, 500, 550, 600, 650, 700, 750, 800, 850, and 900 mM, respectively, in 40 μl. (This calculation incorporates the fact that the binding buffer already contains 100 mM NaCl.) Final salt volume additions were 7 μl for each well. Enough master mix was made for 12 reactions, each containing 4 μl 10×TEC binding buffer, 0.4 μl (200 ng) low range MW ladder (NEB), and 23.6 μl Milli-Q water. 28 μl reaction of the master mix were added to each well containing beads and mixed via pipette. The beads were magnetically collected and the supernatants discarded. The beads were then washed with 150 μl TEC wash buffer and the supernatant discarded. 40 μl EB/Tween was added and mixed well to elute, and the eluates were analyzed by on-chip electrophoresis on a BioAnalyzer DNA High Sensitivity chip to assess NaCl size selection.

To analyze the data, global region tables were created: 45-73 (50 bp), 73-96 (75 bp), 96-120 (100 bp), 145-180 (150 bp), 185-245 (200 bp), 245-285 (250 bp), 290-335 (300 bp), 340-390 (350 bp), 465-555 (500 bp), 680-1070 (776 bp). The BioAnalyzer corrected area for these regions was exported, all corrected areas to the area recovered in the 100 mM NaCl bind were normalized and all traces were graphed. As shown in FIG. 11B, electrostatic screening can also affect the binding of DNA molecules to the TEC resins, and can reduce the affinity of smaller nucleic acids and other polyanions for the resin.

### Example 15: NaCl Size Selection

Nucleic acid elution from TEC resins by varying ion concentration was further demonstrated using the following buffers: (1) 10×pH 4.0 binding buffer: 1 M NaOAc (Ambion), 1 M NaCl (Ambion), 0.1% Tween-20 (Enzo Life Sciences), pH 4.0; (2) TEC NaCl wash buffers: 10 mM sodium phosphate (Amresco), at 165, 180, 195, 210, 225, 240, 255, 270, 285, or 300 mM NaCl (Ambion), 0.01% Tween-20 (Enzo Life Sciences), pH 6.0. Buffers were made by making stock NaCl buffers at 165 and 300 mM and mixing for the desired concentration; (3) TEC wash buffer: 10 mM sodium phosphate (Amresco), 0.01% Tween-20 (Enzo Life Sciences), pH 6.0; (4) EB (Biotek) was supplemented with 0.01% Tween-20 (Enzo Life Sciences).

A DNA “smear” sample (pool of fragmented DNA) was created by shearing Coreill sample NA12878 DNA at 150, 350, and 550 bp and equally mixing all sheared samples. The final nucleic acid concentration of the pool was 18.9 ng/μl. The pool was aliquoted and stored at −20° C. until needed.

The pool of fragmented DNA molecules was bound to TEC resin (Cap-His100), and the DNA molecules were size-selectively eluted with increasing concentrations of ions (NaCl) as follows: 5 μl of TEC beads in 1× Bind was aliquoted into a 200 μl Bio-Rad PCR plate. 13.24 μl (250 ng) of the NA12878 DNA fragment pool, 5 μl 10× binding buffer, and Milli-Q water up to 50 μl were added together and mixed via pipette, the beads magnetically collected, and the supernatants discarded. The TEC beads were then washed with 100 μl of TEC Wash buffer mixed via pipette, the beads magnetically collected, and the supernatant discarded. The beads were washed with 16 μl 165 mM wash buffer, mixed on a thermomixer for 30 seconds at 2000 rpm, and spun quickly to collect the beads. The eluate was transferred to a clean well and the process repeated for the remaining NaCl wash buffers, performing washes in order of increasing amounts of salt.

To the eluates, 2 μl of 10×TEC Bind and 2 μl of the beads was added and mixed by shaking for 30 seconds at 2000 rpm, quick spun, the beads magnetically collected, and the supernatant discarded. The TEC beads were then washed with 40 μl TEC Wash buffer, mixed by shaking for 30 seconds at 2000 rpm, spun quickly to collect the beads and the supernatant discarded. The TEC beads were eluted in 10 μl of TEC elute with mixing by shaking for 30 seconds at 2000 rpm, spinning quickly, collecting the beads, and transferring them to a clean well on the plate. 1 μl of each eluate was analyzed by on-chip electrophoresis on a BioAnalyzer High Sensitivity chip, along with 1 μl of 8 ng/μl sheared pool input, and the results shown in FIGS. 12A and 12B. As shown in FIG. 12A, size-dependent elution of DNA molecules from the TEC resin was achieved by electrostatic screening with NaCl at constant pH (6.0), where increasing concentrations of salt removed progressively larger fragments of DNA. The same is shown in FIG. 12B as a gel representation.

### Example 16: Imidazole Size Selection

Nucleic acid elution from TEC resins by varying ion concentration was further demonstrated by varying imidazole concentration. Stock imidazole buffers were prepared as follows. The 500 mM imidazole pH 6.0 buffer was prepared by diluting 1.702 g imidazole (CalBiochem) with 30 ml Milli-Q water and the pH adjusted to 6.0 with 6 N HCl. The volume was adjusted to 49.5 ml and the pH adjusted to 6.0 with 6 N HCl, and the volume brought to 50 ml. The 100 mM imidazole pH 6.0 buffer was prepared by diluting 1 ml 500 mM imidazole buffer with 3.9 ml Milli-Q water. The pH was adjusted to 6.0 with 6 N HCl, and the volume brought to 5 ml. To cover the range of 100-500 mM imidazole (100 μl each), appropriate dilutions of the 500 mM imidazole pH 6.0 buffer and the 100 mM imidazole pH 6.0 buffer were made. The buffers were sealed in a 96-well storage plate and stored at room temperature.

The following buffer sets were used: (1) 10×pH 4.0 binding buffer: 1 M NaOAc (Ambion), 1 M NaCl (Ambion), 0.1% Tween-20 (Enzo Life Sciences), pH 4.0; (2) TEC imidazole wash buffers: at 100, 125, 150, 175, 200, 225, 250, 275, 300, 375, 400, 425, 450, 475, and 500 mM imidazole (CalBiochem), pH 6.0 (buffers were made by making stock pH 6.0 imidazole buffers at 100 and 500 mM and mixing for the desired concentration); (3) TEC wash buffer: 10 mM sodium phosphate (Amresco), 0.01% Tween-20 (Enzo Life Sciences), pH 6.0; (4) EB (Biotek) was supplemented with 0.01% Tween-20 (Enzo Life Sciences).

A pool of sheared DNA was created by shearing Coreill sample NA12878 DNA at 150, 350, and 550 bp and equally mixing all sheared samples. The final nucleic acid concentration of the pool was 18.9 ng/μl. The pool was aliquoted and stored at −20° C. until needed. 5 μl of TEC beads in 1× Bind was aliquoted into a 200 Bio-Rad PCR plate. 13.24 μl (250 ng) of the NA12878 shear pool, 5 μl 10× binding buffer, and Milli-Q water up to 50 μl was added together, mixed via pipette, the beads magnetically collected, and the supernatants discarded. The TEC beads were washed with 100 μl of TEC Wash buffer, mixed via pipette, the beads magnetically collected, and the supernatant discarded. 18 μl 100 mM imidazole buffer was then added, the samples shaken at 2000 rpm for 15 seconds, spun quickly and the beads magnetically collected. The eluate was transferred to a clean well and the process repeated for the remaining buffers, washing in order of increasing amounts of imidazole. 2 μl of each sample was removed and assayed for DNA concentration by Qubit dsDNA HS (Life Technologies).

TABLE 11
Concentration of DNA in imidazole buffer eluate

| Imidazole | Conc |
|---|---|
| Buffer (mM) | (ng/μl) |
| 100 | ND |
| 125 | ND |
| 150 | 0.053 |
| 175 | 0.186 |
| 200 | 0.275 |
| 225 | 0.470 |
| 250 | 0.500 |
| 275 | 0.697 |
| 300 | 0.751 |
| 325 | 0.765 |
| 350 | 0.822 |
| 375 | 0.867 |
| 400 | 0.054 |
| 425 | 0.907 |
| 450 | 0.248 |
| 475 | 0.097 |
| 500 | 0.135 |
| 375 (buffer | ND |
| control) | |

The concentrations from samples in the range from 200-450 mM imidazole (see Table 11) appeared appropriate for running on the Bioanalyzer. To these eluates, 2 μl of 10×TEC Bind and 2 μl of the beads were added, and mixed by shaking for 15 seconds at 2000 rpm, quick spun, the beads magnetically collected, and the supernatants discarded. The TEC beads were then washed with 40 μl TEC Wash buffer, mixed by shaking for 15 seconds at 2000 rpm, spun quickly to collect the beads and the supernatants discarded. The TEC beads were eluted in 8 μl of TEC elute, mixed by pipette, spun quickly to collect the beads, and transferred to a clean well on the plate. 1 μl of each was analyzed by on-chip electrophoresis on a BioAnalyzer High Sensitivity chip and the results are shown in FIG. 13. As indicated by FIG. 13, size-dependent elution of DNA molecules from the TEC resin was achieved by electrostatic screening with imidazole at constant pH (6.0). Like size-dependent elution with NaCl, increasing concentrations of imidazole removed progressively larger fragments of DNA.

### Example 17: Nucleic Acid Capture from FFPE Material with TEC Resins (TEC Express)

A comparison of the nucleic acid extraction efficiency was performed for the “TEC Express” and Covaris truXTRAC DNA FFPE extraction methods using the following buffers: 10×pH 4.0 binding buffer: 1 M NaOAc (Ambion), 1 M NaCl (Ambion), 0.1% Tween-20 (Enzo Life Sciences), pH 4.0. TEC NaCl wash buffer: 10 mM sodium phosphate (Amresco), 100 mM NaCl (Ambion), 0.01% Tween-20 (Enzo Life Sciences), pH 6.0 (made by mixing 0 and 500 mM NaCl buffers). TEC wash buffer: 10 mM sodium phosphate (Amresco), 0.01% Tween-20 (Enzo Life Sciences), pH 6.0. TEC Elute: EB (Biotek) was supplemented with 0.01% Tween-20 (Enzo Life Sciences).

For the TEC Express method, 10-micron FFPE curls were transferred to 1.5 ml screw-cap microfuge tubes. To each tube, 12.5 μl 10× Kapa Express Extract Buffer (Kapa Biosystems), 110.5 μl Milli-Q water, and 2 μl (2 U) Kapa Express Extract Enzyme (Kapa Biosystems) was added and incubated for 60 minutes at 75° C. on an Eppendorf ThermoMixer C shaking at 1000 rpm for 30 seconds every 5 minutes. The samples were removed from the heat and quick spun to collect any evaporated liquid, and allowed to cool on ice for 3 minutes. For filtered samples, the entire volume was transferred to a 350 μl AcroPrep Advance 96 Filter Plate with 3 μm GF/0.2 μm Supor (Pall Life Sciences, 8075) with a 350 μl conical bottom collection plate. To clarify the samples, full vacuum was applied until all liquids have gone through the filter (˜5 seconds). 125 μl Milli-Q water was added to each filter well and vacuum was applied until the wash had been pulled through the filter. (This step can be skipped for unfiltered samples.)

To each aliquot, 30 μl 10×TEC Bind and 25 μl of Cap-His100 beads was added, mixed well, the beads magnetically collected and the supernatants discarded. The beads were washed with 2×200 μl TEC NaCl Wash Buffer followed by 200 μl TEC Wash Buffer, and the samples eluted in 55 μl TEC elute, by incubating at 60° C. for 5 minutes (with a 30 second 1,000 rpm shake at the start of the incubation).

For an additional 2 samples, nucleic acids were extracted from 10-micron FFPE curls using the truXTRAC DNA FFPE kit (Covaris) according to the manufacturer's protocol, with the following modifications: The proteinase K digestion was performed for 1.5 hours at 60° C. in a Hybex oven. (Note that the digestion temperature was increased from 56° C. to account for thermal loss in the Hybex.) The cross-links were reversed at 80° C. for 1 hour in a Hybex oven already at temperature. All the samples look relatively cloudy and in some a little particulate was visible. Each sample was eluted in BE with 2×50 μl aliquots and all eluates from the same sample were combined.

For DNA Quantitation, a 1:50 dilution of RNase A (Qiagen) was made in TE pH 8.0 (Amresco). 8 μl of the diluted RNase nuclease was added to 2 μl of each TEC Express or Covaris truXTRAC sample and incubated at 37° C. for 30 minutes. 190 μl of Qubit dsDNA High Sensitivity working solution (Life Technologies) was added and the samples were quantified on the Qubit fluorimeter, and the results are presented in Table 12.

TABLE 12
DNA Quantitation from FFPE Materials

| | | | | | | Ave | |
|---|---|---|---|---|---|---|---|
| | | | Elution | | Norm | Norm | |
| | Input | Conc | Vol | Mass | Mass | Mass | |
| Sample | (mg) | (ng/μl) | (μl) | (ng) | ng/mg | ng/mg | SD |
| Covaris | 1.8 | 3.04 | 100 | 304.0 | 168.9 | 171.8 | 4.1 |
| truXTRAC - | | | | | | | |
| B1 | | | | | | | |
| Covaris | 1.7 | 2.97 | 100 | 297.0 | 174.7 | | |
| truXTRAC - | | | | | | | |
| B2 | | | | | | | |
| TEC | 0.6 | 3.71 | 55 | 204.0 | 340.1 | 255.6 | 119.4 |
| Express - | | | | | | | |
| B3 | | | | | | | |
| TEC | 0.8 | 2.49 | 55 | 137.0 | 171.2 | | |
| Express - | | | | | | | |
| B4 | | | | | | | |
| TEC Express | 0.7 | 2.59 | 55 | 142.5 | 203.5 | 196.4 | 10 |
| Filter - B5 | | | | | | | |
| TEC Express | 0.7 | 2.41 | 55 | 132.6 | 189.4 | | |
| Filter - B6 | | | | | | | |

For DNA Size Analysis, the DNA samples obtained from both the TEC Express and Covaris truXTRAC methods were prepared and analyzed on the Fragment Analyzer (Advanced Analytics) according to the manufacturer's protocol, and the results shown in FIG. 14A. The Covaris truXTRAC and TEC Express filtered samples are indicated in the figure. As can be seen from FIG. 14A, the TEC Express method yielded higher quality (less fragmented) DNA than the Covaris truXTRAC kit. Moreover, the TEC Express method takes only 1.5 hours to perform, and provides higher recovery rates (measured as ng output per mg input) compared with the Covaris truXTRAC FFPE DNA extraction method, which typically takes 5-6 hours to perform.

An additional experiment was performed as described above to demonstrate the ability of the TEC Express method to size-separate small RNA molecules, except that the RNase A step was not performed. Small RNA molecules captured and eluted from the FFPE samples were analyzed by on-chip electrophoresis on a BioAnalyzer High Sensitivity chip, and the results are shown in FIG. 14B. As shown in FIG. 14B, the TEC Express method can separate RNA molecules as small as four nucleotides in length, and distinguish RNA molecules of varying sized in a range of about 4 through at least 150 nucleotides in length. These results show that the TEC Express method is capable of isolating and size-separating nucleic acid molecules of small size, such as small interfering RNAs (siRNAs) and micro-RNAs (miRNAs).

### Example 18: TEC Express and Covaris truEXTRAC Sequencing

DNA samples from TEC Express and Covaris truEXTRAC DNA FFPE extractions obtained in Example 17 were converted to sequencing libraries using the TruSeq Nano library prep kit (Illumina) following the manufacturer's protocol, and using identical parameters for each extraction sample. Samples were pooled and sequenced to a coverage of 30× (−200 billion reads) on the HiSeq 2500 platform (Illumina). Sequencing statistics were collected with standard informatics tools. The sequencing data from this analysis is shown in Table 13. As shown by the data, for similar sequencing depth the TEC Express samples showed higher alignment rates, more “reads” properly paired, and fewer orphaned (singleton) reads compared to sequencing libraries from the same FFPE curl extracted with the Covaris truXTRAC DNA FFPE kit.

TABLE 13
TEC Express and Covaris truEXTRAC Sequencing Data

| TEC Express | Covaris truXTRAC | Sequencing Statistic |
|---|---|---|
| 201300872 | 218153586 | Total Reads |
| 9969545 | 10872725 | Duplicate Reads |
| 5.0% | 5.0% | % Duplicate Reads |
| 176887802 | 177003881 | Mapped Reads |
| 87.9% | 81.1% | % Mapped Reads |
| 87.2% | 80.1% | % Mapped De-duplicated Reads |
| 40303549 | 36423659 | Softclipped Mapped Reads |
| 22.8% | 20.6% | % Softclipped Mapped Reads |
| 37462856 | 33931875 | Softclipped De-duplicated |
| | | Mapped Reads |
| 22.4% | 20.4% | % Softclipped De-duplicated |
| | | Mapped Reads |
| 201300872 | 218153586 | Paired in Sequencing |
| 100650436 | 109076793 | Read 1 |
| 100650436 | 109076793 | Read 2 |
| 169899162 | 161599530 | Properly Paired |
| 84.4% | 74.1% | % Properly Paired |
| 3136464 | 5273495 | Singletons |
| 1.6% | 2.4% | % Singletons |

### Example 19: NaCl Size Selection with 2-AEP TEC Resin

Nucleic acid elution from (2-aminoethyl)pyridine TEC resins by varying ion concentration was demonstrated with the following buffers: 10×TEC Binding Buffer: 1 M NaOAc (Ambion), 1 M NaCl (Ambion), 0.1% Tween-20 (Enzo Life Sciences), pH 4.0; TEC NaCl Wash Buffers pH 5.0: 10 mM sodium phosphate (Amresco), at 225, 251, 276, 285, 294, 303, 313, 322, 331, or 340 mM NaCl (Ambion), 0.01% Tween-20 (Enzo Life Sciences), pH 5.0. (Buffers were made by making stock NaCl buffers at 225 and 340 mM and mixing for the desired concentration.) TEC Wash Buffer pH 5.0: 10 mM sodium phosphate (Amresco), 0.01% Tween-20 (Enzo Life Sciences), pH 5.0; EB (Biotek) was supplemented with 0.01% Tween-20 (Enzo Life Sciences).

A DNA smear sample was created by shearing Coreill sample NA12878 DNA at 150, 350, and 550 bp and equally mixing all sheared samples. The final nucleic acid concentration of the pool was 18.9 ng/μl. The pool was aliquoted and stored at −20° C. until needed. 5 μl of Cap-2AEP100 beads in 1× Bind was aliquoted into a 200 μl Bio-Rad PCR plate. 6.61 μl (125 ng) shear pool DNA, 2.5 μl 10×TEC Binding Buffer, and 10.89 μl Milli-Q water was added together, mixed well via pipette and incubated at room temperature for 1 minute. The beads were magnetically collected and the supernatant discarded. The beads were then washed with 25 μl TEC Wash Buffer pH 5.0, mixed via pipette, magnetically collected, and the supernatant discarded.

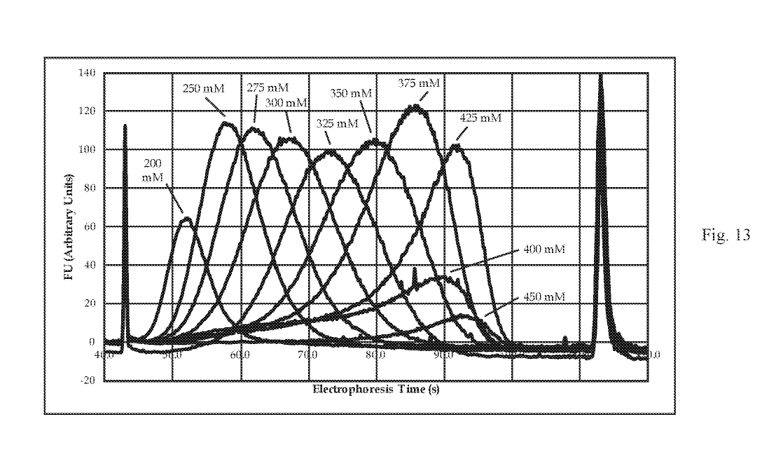

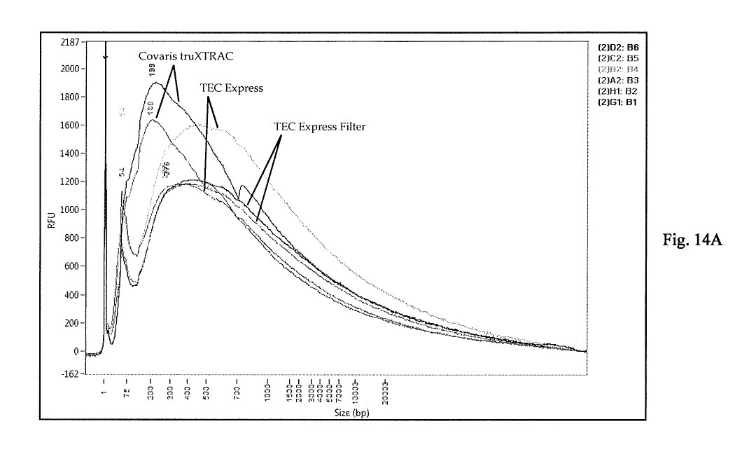

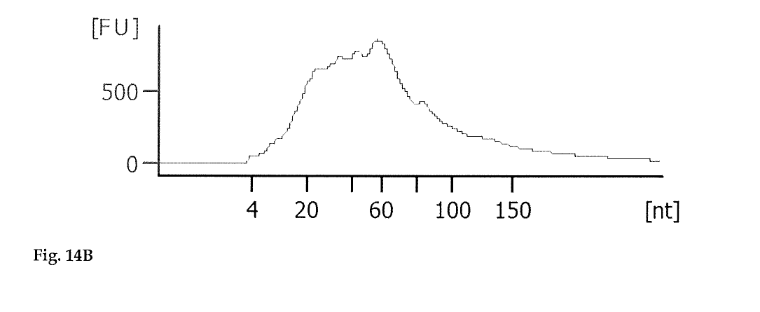

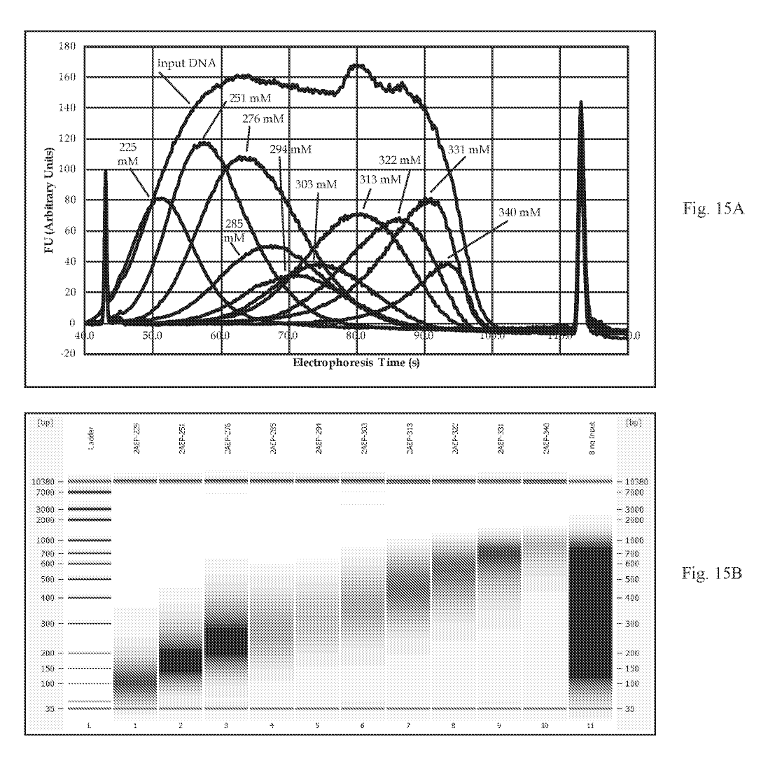

The beads were sequentially washed with 8 μl of TEC NaCl Wash Buffer pH 5.0, starting with the lowest salt concentration (225 mM) and moving to the next higher, and mixed via pipette with no quick spins in between washes. The supernatants were saved for analysis. After the final salt wash (340 mM), the beads were re-suspended in 8 μl TEC Elute, mixed via pipette, magnetically collected, and the eluate was removed/saved. 1 μl of 10×TEC Bind and 1 μl of the beads was added to the elutates, mixed by shaking for 30 seconds at 2000 rpm, quick spun and the beads collected. The supernatant was discarded. The beads were then washed with 25 μl TEC Wash Buffer pH 5.0, mixed by shaking for 30 seconds at 2000 rpm, quick spun and collected. The supernatant was again discarded. The beads were then eluted with 5 μl of TEC elute, mixed by shaking for 30 seconds at 2000 rpm, quick spun, collected, and transferred to a clean well on the plate. 1 μl of each eluate was analyzed by on-chip electrophoresis on a BioAnalyzer High Sensitivity chip, along with 1 μl of 8 ng/μl sheared pool input, to determine the fragment lengths eluted at each NaCl concentration. The results of the size distribution analysis by BioAnalyzer are shown in FIG. 15A, and the same results are shown as a gel representation in FIG. 15B. As shown in FIGS. 15A and 15B, size-dependent elution from the 2AEP resin was achieved by electrostatic screening with NaCl, where increasing concentrations of salt removed progressively larger fragments of DNA.

### Example 20: Comparing Capture Capabilities of CM, Cap-2AEP100, and Cap-His100 Beads

As illustrated above, Cap-His100 TEC beads work well for nucleic acid capture and size selection. To test whether is it possible to leverage the inherent ionizable groups on the surface of the dsMG-CM beads to effect nucleic acid capture and size selection, this experiment compared the capture and size selection capabilities of CM, Cap-2AEP100, and Cap-His100 beads.

This experiment used the Thermo Carboxy-Modified SpeedBeads (CM beads) as a magnetic bead base. The beads were tested to confirm that the bead solution (lot#141291) was 5.16% solids (51.6 mg/ml) and the carboxyl content was 0.4197 mmol/g.

97 μl (5 mg) of the beads were transferred to a clean 1.5 ml microfuge tube. In addition, 1 ml of Cap-2AEP100 was divided into 2×500 μl aliquots. The beads were magnetically collected and the supernatants discarded. The beads were washed with 3×500 μl 1×TEC Binding Buffer: 1 M NaOAc (Ambion), 1 M NaCl (Ambion), 0.1% Tween-20 (Enzo Life Sciences), pH 4.0. All beads were resuspended in 480 μl of 1×TEC Binding Buffer for a final volume of 500 μl. It was observed that the CM and Cap-2AEP100 resins settled much faster than Cap-His100 in low pH binding buffer.

For bead capture reactions, the following buffers were used: 10×TEC Binding Buffer: 1 M NaOAc (Ambion), 1 M NaCl (Ambion), 0.1% Tween-20 (Enzo Life Sciences), pH 4.0; TEC Wash Buffer pH 6.0: 10 mM sodium phosphate (Amresco), 0.01% Tween-20 (Enzo Life Sciences), pH 6.0; TEC Wash Buffer pH 5.0: 10 mM sodium phosphate (Amresco), 0.01% Tween-20 (Enzo Life Sciences), pH 5.0; TEC Elute: EB (Biotek) was supplemented with 0.01% Tween-20 (Enzo Life Sciences).

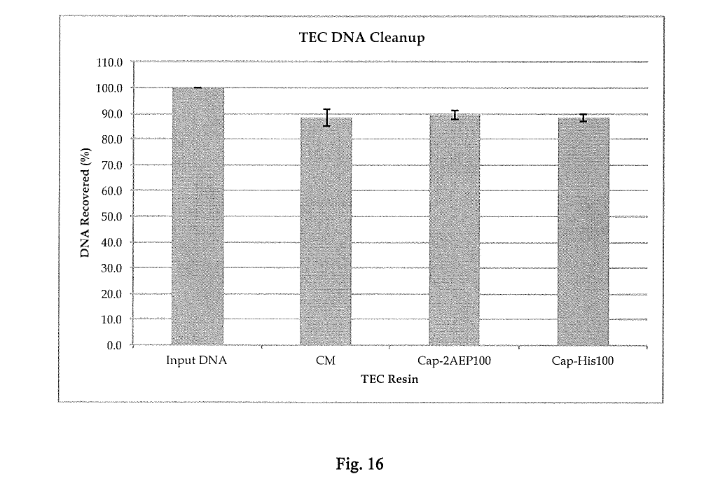

3×2.5 μl of CM, Cap-2AEP100, and Cap-His100 beads were aliquoted into a Bio-Rad PCR plate. To each well, 4 μl of a transposon library (02.10.15, BHL044) was added at 60.4 ng/μl (241.6 ng each). A master mix of 25 μl 10×TEC Binding Buffer and 160 μl Milli-Q water was made and 18.5 μl of the mix was added to each well. The plate was sealed and mixed at 2000 rpm for 30 seconds on an Eppendorf ThermoMixer C. The plate was spun quickly, the beads magnetically collected, and the supernatants discarded. 40 μl of TEC Wash Buffer pH 5.0 was added to the CM and Cap-2AEP-100 resins, and 40 μl of TEC Wash Buffer pH 6.0 was added to the Cap-His100 wells. The plate was sealed and mixed at 2000 rpm for 30 seconds on an Eppendorf ThermoMixer C, quick spun, the beads magnetically collected, and the supernatants discarded. 15 μl of TEC Elute was added to each well and the plate was sealed and mix at 2000 rpm for 30 seconds on an Eppendorf ThermoMixer C. The beads were magnetically collected before sampling for Qubit analysis. Roughly 16 μl of eluate could generally be collected from each well. For recovery quantitation, 2 μl was sampled from each cleanup reaction and 3×1 μl was sampled from the input for Qubit dsDNA HS assay (Life Technologies). A plot of % of DNA recovered is shown in FIG. 16. As shown in FIG. 16, by using buffers tailored to each resin type, all three resins captured about the same amount of DNA (roughly 90% of the input DNA in this assay).

### Example 21: Comparison of Size Selection Capabilities of CM Beads

The ability of carboxy-modified beads to effect size selection of nucleic acids according to the present methods was investigated using Thermo Carboxy-Modified SpeedBeads (CM beads) and the following buffers: 10×pH 4.0 binding buffer: 1 M NaOAc (Ambion), 1 M NaCl (Ambion), 0.1% Tween-20 (Enzo Life Sciences), pH 4.0; TEC NaCl wash buffers: 10 mM sodium phosphate (Amresco), 0-500 mM NaCl (Ambion) in 20 mM steps, 0.01% Tween-20 (Enzo Life Sciences), pH 4.5 or 5.0 (buffers were made by making stock buffers at 0 and 500 mM and mixing for the desired concentration); TEC wash buffers: 10 mM sodium phosphate (Amresco), 0.01% Tween-20 (Enzo Life Sciences), pH 4.5 or 5.0; EB (Biotek) was supplemented with 0.01% Tween-20 (Enzo Life Sciences).

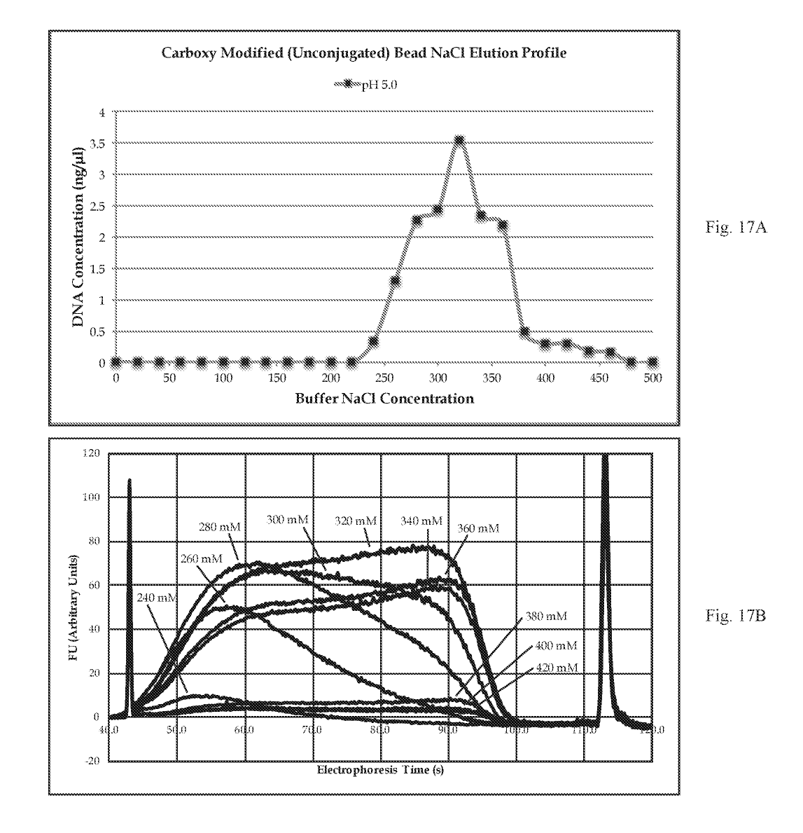

A DNA shear pool was created with a mixture of NA12878 DNA sheared at 150, 350, and 550 bp. The final nucleic acid concentration of the pool was 18.9 ng/μl. The pool was aliquoted and stored at −20° C. until needed. 2×5 μl of CM beads were aliquoted in 1×TEC binding buffer into a 200 μl Bio-Rad PCR plate. To each aliquot, 13.24 μl (250 ng) of the NA12878 shear pool and 5 μl 10× binding buffer was added with Milli-Q water up to 50 μl, mixed via pipette, the beads magnetically collected, and the supernatants discarded. The beads were washed with 100 μl of TEC Wash buffer (one at pH 4.5, the other at pH 5.0) by mixing via pipette, the beads magnetically collected, and the supernatant discarded. The beads were then washed with 16 μl mM NaCl wash buffer, mixed on a thermomixer for 30 seconds at 2000 rpm, quick spun, the beads magnetically collected, and the eluate transferred to a clean well. The process was repeated for the remaining NaCl wash buffers, performing washes in order of increasing amounts of salt. 1 μl was removed from each eluate and Qubit dsDNA HS quantitation (Life Technologies) was performed. The data is graphed in FIG. 17A. From this figure, it is clear that both pH values work well, but the pH 4.5 sample did not completely elute from the beads at 500 mM NaCl, and the profiles are shifted to higher concentrations compared to previous Cap-His100 pH 6.0 elutions.

The ability of CM beads to perform nucleic acid size selection cleanup was further tested as follows. 12 μl of the 240-420 mM NaCl pH 5.0 eluates were combined with 2 μl of 10×TEC Bind, 2 μl of CM beads, and 4 μl Milli-Q water, mixed by shaking for 30 seconds at 2000 rpm, quick spun to collect the beads, and the supernatant discarded. The beads were washed with 40 μl TEC Wash buffer pH 5.0, mixed by shaking for 30 seconds at 2000 rpm, quick spun to collect the beads, and the supernatant discarded. The samples were eluted in 7.5 μl of TEC Elute, mixed by shaking for 30 seconds at 2000 rpm, quick spun to collect the beads, and transferred to a clean well on the plate. 1 μl of each eluate was run on a BioAnalyzer High Sensitivity chip and the results are shown in FIG. 17B. As indicated in FIG. 17B, the elution profile for each fraction is very broad, and distinct from previously obtained elution profiles of Cap-His100 beads (compare FIG. 17B, for example, with FIG. 12A). Without wishing to be bound by any theory, it appears that the surface modifications of the carboxylic acid beads do not completely replicate the size selection capabilities of TEC resins such as Cap-His100.

### Example 22: Comparison with CHARGESWITCH® Capture Efficiency

To compare the efficiency of TEC resins and CHARGESWITCH® in DNA cleanups, the following buffers were prepared: 10×TEC Binding Buffer (20150210): 1 M NaOAc (Ambion), 1 M NaCl (Ambion), 0.1% Tween-20 (Enzo Life Sciences), pH 4.0; TEC Wash Buffer pH 6.0 (20141112 QC lot): 10 mM sodium phosphate (Amresco), 0.01% Tween-20 (Enzo Life Sciences), pH 6.0; TEC Elute (20141112 QC lot); EB (Biotek) was supplemented with 0.01% Tween-20 (Enzo Life Sciences). The input library used for this experiment was a DNA fragmentation pool (150, 350 and 550 bp of sheared NA12878 DNA) prepared as in the previous Examples, at 18.9

The method for the CHARGESWITCH® cleanup protocol was followed with both CHARGESWITCH® and TEC reagents/beads. In addition, the method for the CHARGESWITCH® cleanup protocol was followed using CHARGESWITCH® beads and TEC buffers (hybrid method). To conduct the cleanup protocols, 6×6 μl (113.4 ng each) of the DNA shear pool was aliquoted into 1.5 ml microfuge tubes. 19 μl Milli-Q water, 25 μl CHARGESWITCH® Purification Buffer (N5) and 10 μl CHARGESWITCH® Magnetic Beads (2.5% solids) were then added to two samples, mixed well via pipette and incubated at room temperature for 1 minute. 39 μl Milli-Q water, 5 μl 10×TEC Binding Buffer, and 10 μl Cap-His100 beads were added to two samples, mixed well via pipette and incubated at room temperature for 1 minute. 39 water, 5 μl 10×TEC Binding Buffer, and 10 μl CHARGESWITCH® beads were added to two the remaining two samples, mixed well via pipette and incubated at room temperature for 1 minute. All beads were magnetically collected for 1 minute and the supernatants discarded.

CHARGESWITCH® beads were washed with 150 μl CHARGESWITCH® Wash Buffer (W12), mixed via pipette, quick spun and the beads magnetically collected for 1 minute. TEC beads and the hybrid method CHARGESWITCH® beads were washed with 150 μl TEC Wash Buffer, mixed via pipette, quick spun, the beads magnetically collected for 1 minute and the supernatants discarded. All samples were then quick spun, and returned them to the magnet and any residual liquid removed.

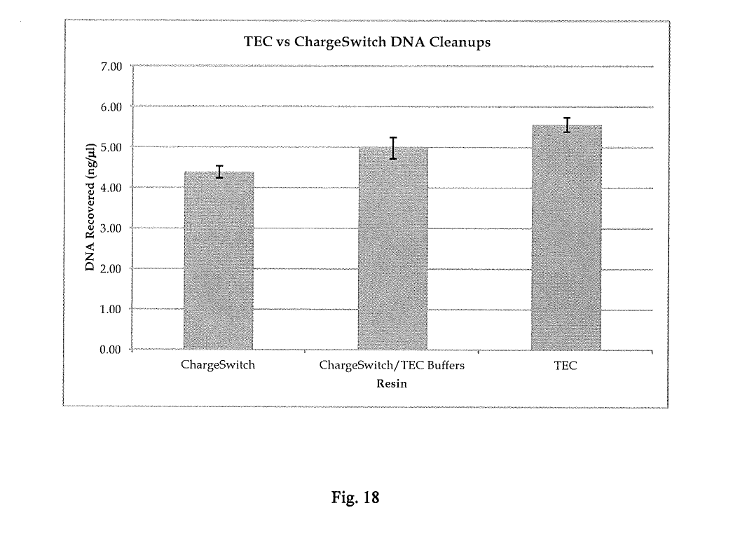

The CHARGESWITCH® beads were resuspended in 25 μl CHARGESWITCH® Elution Buffer, mixed via pipette and incubated at room temperature for 1 minute. The beads were magnetically collected for 1 minute and the eluate was removed/saved. The TEC beads and hybrid method CHARGESWITCH® beads were resuspended in 25 μl TEC Elute, mixed via pipette and incubated at room temperature for 1 minute. The beads were magnetically collected for 1 minute and the eluate removed/saved. All samples were quantified using the Qubit dsDNA HS assay (Life Technologies) using 5 μl of each sample diluted into 195 μl of working solution, and the results are shown in FIG. 18. As indicated in the FIG. 18, The TEC system was more efficient than CHARGESWITCH® in recovering DNA from the samples. In addition, the use of TEC buffers enhanced the DNA recovery from CHARGESWITCH® beads.

### Example 23: Comparison with CHARGESWITCH® Size Selection Capabilities

The size selection capabilities of TEC resins were compared to those of CHARGESWITCH® beads using the following buffers: 10×TEC Binding Buffer (20150210): 1 M NaOAc (Ambion), 1 M NaCl (Ambion), 0.1% Tween-20 (Enzo Life Sciences), pH 4.0; TEC Wash Buffer pH 6.0 (20141112 QC lot): 10 mM sodium phosphate (Amresco), 0.01% Tween-20 (Enzo Life Sciences), pH 6.0; TEC NaCl Wash Buffers pH 6.0 (20141112 QC lot): 10 mM sodium phosphate (Amresco), at 115, 118, 121, 123, 126, 129, 132, 134, 137 or 140 mM NaCl (Ambion), 0.01% Tween-20 (Enzo Life Sciences), pH 6.0; TEC Elute (20141112 QC lot): EB (Biotek) is supplemented with 0.01% Tween-20 (Enzo Life Sciences). The input library used for this experiment was a DNA fragmentation pool (150, 350 and 550 bp of sheared NA12878 DNA) prepared as in the previous Examples, at 18.9 ng/μl.

To perform the size selection experiments, 5 μl of CHARGESWITCH® beads (Life Technologies) was aliquoted into a Bio-Rad 200 μl PCR plate and 6.61 μl (125 ng) shear pool DNA, 2.5 μl 10×TEC Binding Buffer and 10.89 μl Milli-Q water was added, mixed well via pipette and incubated at room temperature for 1 minute. The beads were magnetically collected and the supernatant discarded. The beads were then washed with 25 μl TEC Wash Buffer, mixed via pipette, the beads magnetically collected, and the supernatant discarded.

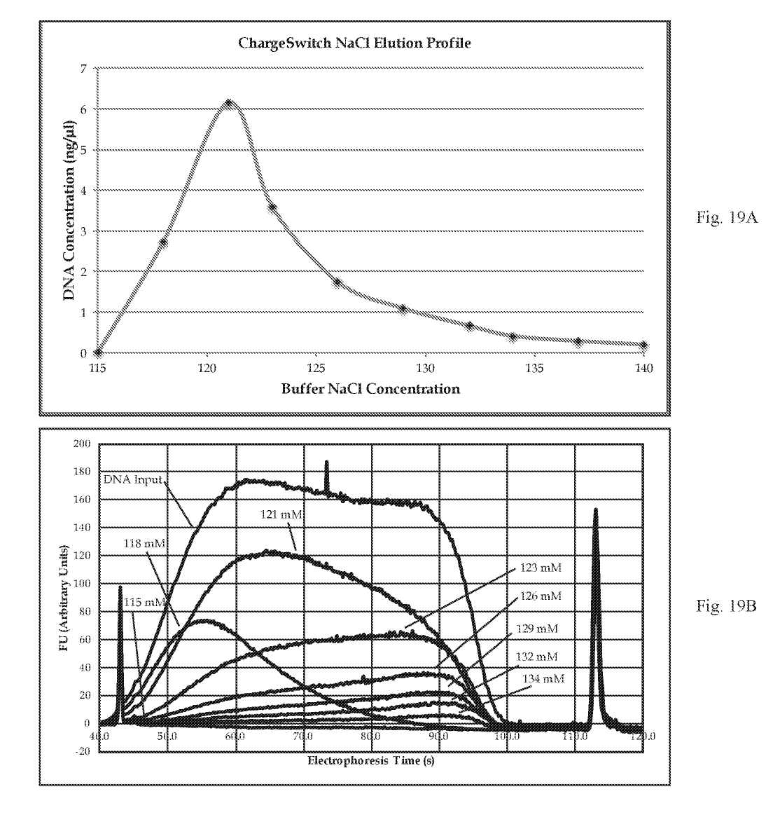

Next the beads were sequentially washed with 8 μl of TEC NaCl Wash Buffer, starting with the lowest salt concentration and moving to the next higher, by mixing via pipette with no quick spins in between washes. The supernatants were saved for analysis. After the final salt wash, the beads were resuspending in 8 μl TEC Elute, mixed via pipette, the beads magnetically collected and the eluate removed/saved. All samples were quantified using the Qubit dsDNA HS assay (Life Technologies) and the results are shown in FIG. 19A. This analysis shows that the material began eluting from the CHARGESWITCH® beads above 115 mM NaCl, and elution was essentially complete by 135 mM NaCl.

2 μl of each sample was then diluted into 198 μl of working solution. To each eluate, 1 μl of CHARGESWITCH® beads and 1 μl of TEC Binding Buffer was added, mixed well, the beads magnetically collected and the supernatants discarded. The beads were washed with 20 μl TEC Wash Buffer, magnetically collected and the supernatant discarded. The samples were eluted by re-suspending the beads in 6 μl TEC Elute, and were analyzed by loading 1 μl on the Bioanalyzer DNA High Sensitivity chip for on-chip electrophoresis to determine the fragment lengths eluted at each NaCl concentration. The results are shown in FIG. 19B. Like the carboxy-modified beads tested in the example, FIGS. 19A and 19B show that CHARGESWITCH® beads were able to capture DNA, but were unable to effectively perform size selection.

### Example 24: Comparison of Nucleic Acid Capture Efficiency for TEC and CM Beads in SDS

The relative efficiency of nucleic acid capture by TEC beads and carboxy-modified beads unconjugated with TEC ligands (CM beads) in the presence of sodium dodecyl sulfate was tested using the following buffers: 10×TEC Binding Buffer: 1 M NaOAc (Ambion), 1 M NaCl (Ambion), 0.1% Tween-20 (Enzo Life Sciences), pH 4.0; TEC Wash Buffer pH 6.0: 10 mM sodium phosphate (Amresco), 0.01% Tween-20 (Enzo Life Sciences), pH 6.0; TEC Wash Buffer pH 5.0: 10 mM sodium phosphate (Amresco), 0.01% Tween-20 (Enzo Life Sciences), pH 5.0; TEC Elute; EB (Biotek) was supplemented with 0.01% Tween-20 (Enzo Life Sciences).

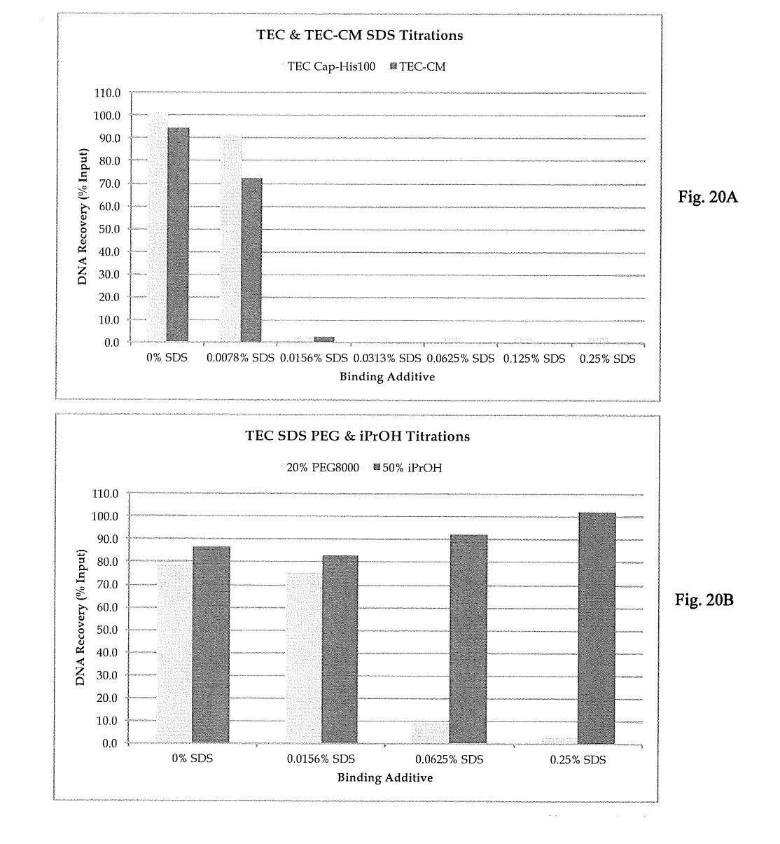

Nucleic acid capture was effected as follows: 7×2.5 μl of TEC Cap-His100 and 7×2.5 μl of CM beads were aliquoted into a 200 μl Bio-Rad PCR plate. To one aliquot of each bead type, 2.5 μl of SDS (Amresco) were added at the following concentrations: 2.5%, 1.25%, 0.625%, 0.3125%, 0.1563%, 0.0781% and 0%. Final SDS concentrations in the binding reaction were 10-fold less. A 15-reaction master mix was created by combining 37.5 μl 10×TEC Binding Buffer, 30 μl PCR-amplified DNA library (1464 ng) and 232.5 μl Milli-Q water, and mixing well. 20 μl of this master mix was added to each bead sample for a 25 μl final volume. The samples were mixed for 30 seconds at 2000 rpm in an Eppendorf ThermoMixer C, and incubated at room temperature for 5 minutes. The samples were then quick spun, the beads magnetically collected, and the supernatants were discarded. The beads were washed with 2×25 μl TEC Wash Buffer, using pH 6.0 for Cap-His100, and pH 5.0 for the CM beads. The DNA was eluted in 15 μl TEC elute, the beads magnetically collected and 5 μl of the eluate was removed for analysis via Qubit dsDNA HS Assay (Life Technologies). The results are shown in FIG. 20A. As indicated in FIG. 20A, both TEC and CM beads were able to capture DNA in the presence of up to 0.0078% SDS (although the TEC beads were more efficient), but neither bead type was able to appreciably capture DNA in SDS concentrations from 0.0156% to 0.25%. These data show that an amphiphilic detergent molecule like SDS inhibits the binding of polyanions to the surface of TEC and CM beads.

However, the addition of certain compounds to the binding environment can mitigate the inhibitory effect of SDS on polyanion binding to a TEC resin. To show this, DNA was bound to TEC beads with either PEG8000 or isopropyl alcohol (iPrOH) in the presence of different SDS concentrations, using the following buffers: 10×TEC Binding Buffer (20150210): 1 M NaOAc (Ambion), 1 M NaCl (Ambion), 0.1% Tween-20 (Enzo Life Sciences), pH 4.0; TEC Wash Buffer pH 6.0 (20141112 QC lot): 10 mM sodium phosphate (Amresco), 0.01% Tween-20 (Enzo Life Sciences), pH 6.0; TEC Elute (20141112 QC lot): EB (Biotek) was supplemented with 0.01% Tween-20 (Enzo Life Sciences).

To effect the DNA binding, 8×2.5 μl of TEC Cap-His100 beads were aliquoted into a 200 μl Bio-Rad PCR plate. To two aliquots of each bead type, 2.5 μl of SDS (Amresco) was added at the following concentrations: 2.5%, 0.625%, 0.1563%, and 0%. Final concentrations in the binding reaction were 10-fold less. A 4.5-reaction iPrOH master mix was created by combining 11.25 μl 10×TEC Binding Buffer, 4.5 μl PCR DNA (220 ng), 56.25 μl 2-propanol (Amresco) and 18 μl Milli-Q water and mixing well. 20 μl of this master mix was added to each bead sample for a 25 μl final volume having 50% iPrOH. A 4.5-reaction PEG8000 master mix was created by combining 11.25 μl 10×TEC Binding Buffer, 4.5 μl PCR DNA (220 ng), 45 μl PEG8000 (NEB), and 29.25 μl Milli-Q water and mixing well. 20 μl of this master mix was added to each bead sample for 25 μl final volume having 20% PEG8000. The reactions were mixed for 30 seconds at 2000 rpm in an Eppendorf ThermoMixer C, and the beads incubated at room temperature for 5 minutes. The samples were then quick spun, the beads magnetically collected, and the supernatants were discarded.

The beads were washed with 2×100 μl TEC Wash Buffer, using pH 6.0. For the washes, the buffer was simply overlayed with the beads still on the magnet, gently pipetted up and down a few times, and the wash buffer let sit for 1 minute. The wash buffer was then discarded. The DNA was eluted in 15 μl TEC elute, the beads magnetically collected and 10 μl of the eluate was removed for analysis via Qubit dsDNA HS Assay (Life Technologies). The results are shown in FIG. 21B. As indicated in FIG. 21B, the DNA binding inhibitory effect of SDS in concentrations up to 0.25% is completely countered if the binding is carried out in a 50% iPrOH buffer. Likewise, the DNA binding inhibitory effect of SDS in concentrations up to 0.0156% is completely countered if the binding is carried out in a 20% PEG8000 buffer. However, DNA binding to TEC beads is still substantially inhibited at higher SDS concentrations, even when carried out in 20% PEG8000 buffer.

### Example 25: Normalization of DNA Concentrations from Multiple Samples

The following experiment was conducted to demonstrate the usefulness of the present compositions and methods for nucleic acid mass normalization from multiple samples. Briefly, the Cap-His100 TEC beads were diluted with “inert” passivated carboxy-modified beads unconjugated with TEC ligands (Cap-CM beads) at different ratios to decrease the binding capacity of the TEC beads, and the resulting “normalization mixtures” were used to capture pre-defined amounts of DNA (5, 10, 20, or 40 ng) from ˜100 ng of a PCR-amplified sequencing library.

The buffers used in this experiment were: 10×TEC Binding Buffer: 1 M NaOAc (Ambion), 1 M NaCl (Ambion), 0.1% Tween-20 (Enzo Life Sciences), pH 4.0; TEC Wash Buffer pH 6.0: 10 mM sodium phosphate (Amresco), 0.01% Tween-20 (Enzo Life Sciences), pH 6.0; and EB from Biotek.

6.4 μl of the TEC beads were diluted with 53.6 μl Cap-CM beads, and the binding capacity of the bead slurry was determined. Additional 2, 4, and 10-fold dilutions of this slurry were made with Cap-CM and volumes were calculated to provide predetermined binding capacities of 5, 10, 20, and 40 ng of nucleic acid. 3×2 μl of each TEC/Cap-CM normalization bead mixture was transferred to a Bio-Rad 200 μl PCR plate, and all volumes were brought up to 5 μl with nuclease-free water (G Biosciences).

The normalization bead mixtures were named as follows:

TEC Norm 1:10 beads: 3×1.67 μl (target of 5 ng nucleic acid binding capacity)—named “Norm-5”;

TEC Norm 1:10 beads: 3×3.33 μl (target of 10 ng nucleic acid binding capacity)—named “Norm-10”;

TEC Norm 1:4 beads: 3×2.56 μl (target 20 ng nucleic acid binding capacity)—named “Norm-20”; and

TEC Norm 1:2 beads: 3×3.04 μl (target 40 ng nucleic acid binding capacity)—named “Norm-40”.

Cap-CM beads alone were used as control.

The input DNA was a PCR-amplified sequencing library (26.7 ng/μl). A 20-reaction master mix of 20 μl 10×TEC Binding Buffer and 80 μl DNA input was made (4 μl or 106.8 ng each reaction), mixed well and 5 μl of this solution was added to each of the wells containing the normalization bead mixtures. Each well was mixed via pipet, the beads magnetically collected and the supernatants discarded. 50 μl of TEC Wash Buffer (pH 6.0) was then added to each well and mixed via pipet, the plate was spun quickly to collect all liquid, the beads magnetically collected and the wash discarded. The beads were resuspended in 5 WEB to elute any bound DNA, mixed via pipet, the plate was spun quickly to collect all liquid, the beads magnetically collected.

All samples were quantified using the Qubit dsDNA HS assay (Life Technologies) using 4 μl of each sample diluted into 196 μl of working solution, and the results are shown in FIG. 21A. The recovery of DNA from each sample was essentially that which was expected based on the calculated normalization bead mixture capture capacity, indicating that the TEC compositions and methods can be used to quickly and efficiently normalize nucleic acid concentrations for subsequent manipulation. The graph of FIG. 21A represents the mass of recovered nucleic acid from each normalization bead mixture vs. the expected recovery (average of 3 independent replicates), and the inset shows the high correlation between the targeted amount of captured DNA and the observed amount returned from the experiment. The present normalization methods using TEC compositions is thus highly reproducible and produce a linear relationship between targeted and observed mass of capture nucleic acid.

In this experiment, the most dilute sample was the 5 ng capture, at 1.1 ng/μl. For further manipulation, the remaining eluates were diluted to this concentration (except for the Cap-CM control beads), and 1 μl of each sample was analyzed by on-chip electrophoresis on a BioAnalyzer DNA High Sensitivity chip to analyze size fragments recovered. The results of this size analysis are shown in FIG. 21B, and it is apparent from this figure that TEC normalization does not result in any observable size bias in the captured nucleic acids.

### Example 26: Optimizations of the TEC System

Example 26 demonstrates “the tunability” of the TEC bead system described herein for different uses and targets.

A. TEC Ligands on Different Bead Types

To explore the use of other bead types in the TEC system, one of the TEC ligands (histamine) was installed on a variety of different bead types and the resulting TEC bead system was ‘tuned’ for high efficiency for each bead type using the methods and techniques described in the examples above.

Histamine was bound to DynaBead MyOne™ carboxylic acid beads (Invitrogen). These beads do not exhibit any background binding. Installation of histamine was successful, and the beads functioned similarly to the original beads for DNA cleanups (Cap-His100) as described in Example 7. Size selection was performed with 10 mM phosphate, 0.01% Tween-20 pH 6 buffer supplemented with NaCl between 525-725 mM final concentration using techniques described in Example 15.

In another experiment, histamine was bound to Sera-Mag Hydrophilic SpeedBeads™ (GE Healthcare). The surface of these beads is hydrophilic instead of hydrophobic. These beads exhibit background binding that was eliminated before installing the TEC ligand by treatment with either acetic anhydride (to produce an acetyl cap) or succinic anhydride (to produce a new carboxylic acid). Installation of histamine using techniques described in Example 2 was successful. These TEC ligand functionalized beads function similarly to the original beads for DNA cleanups as described in Example 7. For the acetyl cap beads, size selection was performed with 10 mM phosphate, 0.01% Tween-20 pH 6 buffer supplemented with NaCl between 280-440 mM final concentration. For the succinic anhydride modified beads, size selection was performed with 10 mM phosphate, 0.01% Tween-20 pH 6 buffer supplemented with NaCl between 450-625 mM final concentration.

The size selection buffers are tailored to the bead chemistry. While the buffer compositions may be different, the performance of all bead types was similar.

B. TEC Binding in Presence of EDTA.

High amounts of EDTA (100 mM or greater) present during the TEC binding was found to prevent DNA from binding to the TEC beads. While not wishing to be bound by theory, the mode of interference was originally thought to be due to EDTA competing for DNA binding to the beads. Without wishing to be bound by theory, it has since been determined that the true mode of inhibition is due to a pH change induced by the EDTA. Dilution of 10×TEC Binding Buffer (1 M sodium acetate, 1 M NaCl, 0.1% Tween-20, pH 4.0) to 1× concentration was found to result in a final pH of 4.25. Performing the same dilution with a final concentration of 50 mM EDTA (pH 8) resulted in a binding pH of 5.15. Performing the same dilution with a final concentration of 75 mM EDTA (pH 8) resulted in a binding pH of 5.56. Performing the same dilution with a final concentration of 100 mM EDTA (pH 8) resulted in a binding pH of 5.91.

DNA binds efficiently in 50 mM and 75 mM EDTA, and is reduced (<2% binding) in 100 mM EDTA (or greater).

It was discovered that diluting 10×TEC Binding Buffer in the presence of 100 mM EDTA (pH 8) and 50 mM sodium acetate (pH 4) results in a binding pH of 5.11, similar to the pH achieved with the use of 50 mM EDTA (pH 8). Binding reactions performed in the presence of 1×TEC Binding Buffer plus 100 mM EDTA (pH 8) and 50 mM sodium acetate (pH 4) performed as efficiently as reactions without any EDTA at all. The increase in pH, rather than molecular competition, caused by 100 mM EDTA or greater is believed to be responsible for the reduced binding in the presence of EDTA. Thus, the TEC binding buffers can be diluted in the presence of high (100 mM or greater) amounts of EDTA provided that pH adjustments are made.

C. Variations of Binding Buffer Compositions

As described above, the original version of 10×TEC Binding Buffer is 1 M sodium acetate, 1 M NaCl, 0.1% Tween-20, pH 4.0. Binding of DNA and RNA is highly efficient in this buffer. However, the removal of the NaCl from the binding buffer results in a loss (˜30%) of binding efficiently of the beads. Without wishing to be bound by theory, the acetate in the buffer may be forming a weak complex with the imidazole ring of the histamine, which in turn reduces the affinity of DNA for the beads. It is theorized that the salt acts as an ion pairing partner for the acetate and prevents it from interacting with the beads. Thus, in most embodiments, the presence of NaCl in the TEC binding buffers is deemed to be necessary for high efficiency binding when an acetate is also part of the binding buffers.

In another buffer composition, citrate was employed as a binding buffer reagent as follows. In one embodiment, a 10× Citrate Binding Buffer consists of 1 M citric acid, 1 M NaCl, 0.1% Tween-20 pH 4.0. Binding of DNA to beads using this citrate binding buffer was found to be highly efficient, both in the presence and absence of NaCl. Thus, in certain embodiments, a citrate compound may be used in the binding buffer compositions described herein instead of an acetate compound.

D. Genomic DNA (gDNA) TEC Capture

As indicated above, for gDNA capture, the TEC binding method can be modified compared to that used for smaller fragment capture (as described in Example 7). When TEC beads bind to very large DNA (i.e., in excess of 10,000 base pairs), they tend to clump together and become particulate. The occurrence of these particulates is indicative of the presence of such large, intact DNA. Because the DNA is interwoven with many beads, the large DNA adheres more strongly to the beads. Thus elution from the beads requires optimization that is slightly different than that described herein for smaller DNA fragments. Elution with standard DNA elution buffers (TE, Tris pH 8.5) returns ˜60% of gDNA. However, raising the pH of the elution buffer to above pH 8.5, or above pH 9 can significantly increase the return of gDNA, up to −90%. Among buffers that are useful as elutions buffers in this context are in certain embodiments, elutions with 10 mM Tris pH 9 or elutions with 10 mM NaOH, or other elutions buffers of similar high pH.

In still another embodiment, the efficiency of capture of very large DNA by the TEC system can be improved by small modifications to the TEC beads. In one embodiment, the Cap-His100 bead is passivated with acetic anhydride, which adds a moderately hydrophobic acetyl cap to the beads. In another embodiment modified for large DNA, these beads are passivated with succinic anhydride, which adds additional negatively charged carboxylic acids to the surface of the beads. This modification enhances elution of gDNA. In one embodiment, increasing the charge-charge repulsion from the bead surface aides in the elution of very large DNA from the beads.

E. Handling Chemical Incompatibilities in Extraction

Primarily for extraction purposes, certain chemical incompatibilities can be adjusted in the TEC system. As one example described above, the inhibitory characteristics of SDS can be mitigated by the use of isopropyl alcohol. In another embodiment, the use of guanidinium (HCl or thiocyanate) are likely to be inhibitory at certain levels. This inhibition may be counteracted by the use of isopropyl alcohol. In still another modification, replacement or supplementation of the SDS or other detergents in the extraction buffers may be counteracted by use of urea, a common denaturant that is compatible with TEC up to high concentrations (at least 4-5 M). Still other modifications may be made in the TEC system based on the particular uses involved.

All patents, patent applications, particularly priority application U.S. application Ser. No. 15/097,781, and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

1. A method for separating polyanions according to size from a sample comprising:
contacting a sample which comprises polyanions of different sizes with a solid support to which is covalently bound a plurality of tunable electrostatic capture (TEC) ligands, wherein the TEC ligands comprise an ionizable ligand which is positively charged at a pH less than a pKa of the ionizable ligand and neutral at a pH greater than the pKa of the ionizable ligand; said support having been contacted with a TEC capture buffer having a pH less than the pKa of the ionizable ligand, which causes the TEC ligands to have a positive charge, wherein the sample is contacted under conditions which allow the TEC ligands to reversibly bind polyanions of different sizes in the sample to the TEC ligands having a positive charge; and
releasing the polyanions from the TEC ligands according to size by contacting the solid support with at least a first TEC release buffer having an ion concentration suitable for releasing polyanions having a first size from the TEC ligands, wherein polyanions having a second size greater than the first size remain bound to the TEC ligands.

2. The method according to claim 1, further comprising:
providing a plurality of tunable electrostatic capture (“TEC”) ligands covalently bound to a solid support, wherein the TEC ligands comprise an ionizable ligand which is positively charged at a pH less than a pKa of the ionizable ligand and neutral at a pH greater than the pKa of the ionizable ligand;
causing the TEC ligands to have a positive charge by contacting the solid support with a TEC capture buffer having a pH less than the pKa of the ionizable ligand.

3. The method of claim 1, further comprising contacting the solid support with at least a second TEC release buffer having a second ion concentration greater than the ion concentration of the first TEC release buffer, wherein the polyanions having a second size greater than the first size are released from the TEC ligands.

4. The method of claim 3, further comprising contacting the solid support with at least one additional TEC release buffer having an additional ion concentration greater than the ion concentration of the second or preceding additional TEC release buffer, wherein the polyanions having a size greater than the second size are released from the TEC ligands; and wherein each additional TEC release buffer permits release of progressively larger polyanions from the TEC ligands.

5-7. (canceled)

8. The method of claim 1, wherein the pH of each TEC release buffer is less than about 7.

9. (canceled)

10. The method of claim 1, wherein the ionizable ligand is histamine, 4-(2-amino)pyridine, 3-(2-amino)pyridine, 2-(2-amino)pyridine, pyridine or imidazole.

11. (canceled)

12. The method of claim 1, wherein the solid support further comprises free carboxylic acid functional groups and the ratio of free carboxylic acids to TEC ligand bound to the solid support is (a) between about 1 to 9 or (b) about 1 to 3.

13. The method of claim 12, wherein the ratio of free carboxylic acids to TEC ligand bound to the solid support is (c) between about 1 to 3 or (d) between about 1 to 1.

14. The method of claim 1, wherein:
(a) the ion concentration of each TEC release buffer is about 1000 mmole/L or less;
(b) a molar concentration of ion in the first TEC release buffer is at least about 10 mmole/L less than a molar concentration of ion in the second TEC release buffer; or
(c) a molar concentration of ion in the second TEC release buffer is at least about 10 mmole/L less than a molar concentration of ion in a subsequent additional TEC release buffer.

15-16. (canceled)

17. The method of claim 1, which is performed using a kit comprising:
(a) a plurality of tunable electrostatic capture (“TEC”) ligands covalently bound to a solid support, wherein the TEC ligands comprise an ionizable ligand which is positively charged at a pH less than the pKa of the ionizable ligand and neutral at a pH greater than the pKa of the ionizable ligand;
(b) a TEC capture buffer having a pH less than the pKa of the ionizable ligand; and
(c) a set of TEC buffers comprising:
i. a TEC wash buffer having a pH greater than that of the capture buffer but less than that of the ionizable ligand on the TEC support;
ii. a first TEC release buffer suitable for releasing polyanions having a first size, the first TEC release buffer having a first ion concentration,
iii. a second TEC release buffer suitable for releasing polyanions having a size greater than the first size, the second TEC release buffer having an ion concentration greater than the ion concentration of the first TEC release buffer; and
iv. a TEC elution buffer having a pH greater than that of the ionizable ligand on the TEC support.

18. The method of claim 17, wherein the kit further comprises at least one additional TEC release buffer suitable for releasing polyanions having an additional size greater than the second size, each said additional buffer having an ion concentration greater than the ion concentration of the second TEC release buffer and different from the ion concentration of any other additional TEC release buffer in the kit.

19. The method of claim 17, wherein in the kit:
(a) the pH of the TEC capture buffer or TEC release buffer is less than about 7;
(b) the pH of the TEC capture buffer is between about 4.0 and about 5.0;
(c) the ionizable ligand is a heterocyclic amine; or
(d) the ionizable ligand is selected from the group consisting of histamine, 4-(2-amino)pyridine, 3-(2-amino)pyridine, 2-(2-amino)pyridine, pyridine and imidazole.

20-22. (canceled)

23. The method of claim 17, wherein the solid support further comprises at least one of:
(a) free carboxylic acid functional groups;
(b) a ratio of free carboxylic acids to bound TEC ligand between about 1 to 9 and about 1 to 3; and
(c) a ratio of free carboxylic acids to bound TEC ligand is between about 1 to 3 and about 1 to 1.

24. The method of claim 17, wherein in the kit:
(a) the ion concentration of the TEC release buffers is about 1000 mmole/L or less;
(b) the ion concentration in the first TEC release buffer is at least about 10 mmole/L less than the ion concentration in the TEC release second buffer; or
(c) the ion concentration in the second TEC release buffer is at least about 10 mmole/L less than the ion concentration in the third TEC release buffer.

25-26. (canceled)

27. The method of claim 17, wherein the kit further comprises at least one of:
(a) instructions for preparing the TEC capture buffer;
(b) instructions for preparing one or more TEC release buffers; and
(c) instructions for size separation of polyanions using the TEC capture buffer and one or more TEC release buffers.

28. A method of normalizing a concentration of polyanions from a plurality of samples comprising:
(a) providing a plurality of samples comprising polyanions, wherein at least one of the plurality of samples has a different concentration of polyanions than the other samples;
(b) providing, for each of the plurality of samples, a substantially similar amount of a solid support covalently bound to a plurality of tunable electrostatic capture (“TEC”) ligands, wherein the TEC ligands comprise an ionizable ligand which is positively charged at a pH less than a pKa of the ionizable ligand and neutral at a pH greater than the pKa of the ionizable ligand;
(c) capturing an amount of polyanions in each of the plurality of samples by mixing the fixed amount the solid supports with each of the plurality of samples in a TEC capture buffer under substantially similar conditions to form a plurality of mixtures, wherein each of the plurality of mixtures has a pH less than the pKa of the ionizable ligand under conditions to allow the TEC ligands to reversibly bind to the polyanions;
(d) isolating the solid supports from each of the plurality of mixtures and keeping each isolated solid support separate; and
(e) releasing the amount of polyanions from the TEC ligands covalently bound to the each of the solid supports isolated in step (d) by contacting the solid supports with a TEC elution buffer having a pH greater than the pKa of the ionizable ligand.

29. (canceled)

30. The method of claim 28, wherein the polyanions comprise nucleic acids, and the amount of polyanions captured in step (c) is predetermined.

31. The method of claim 30, wherein the amount of polyanions captured in step (c) is predetermined, and the samples comprise a concentration of polyanions of at least about the predetermined amount of polyanions captured in step (c).

32. A method of preparing a solid support for binding polyanions comprising:
(a) providing a solid support comprising at least one surface comprising carboxylic acid functional groups;
(b) passivating surface ionizable groups of the solid support;
(c) coupling a plurality of tunable electrostatic capture (“TEC”) ligands to the at least one surface wherein the TEC ligands comprise an ionizable ligand which is positively charged at a pH less than the pKa of the ionizable ligand and neutral at a pH greater than the pKa of the ionizable ligand.

33. (canceled)

34. The method of claim 32, wherein at least one of:
(a) a ratio of free carboxylic acid functional groups to bound TEC ligand after coupling step (c) is between about 1 to 9 and about 1 to 3; or
(b) a ratio of free carboxylic acid functional groups to bound TEC ligand after coupling step (c) is between about 1 to 3 and about 1 to 1.
